(19) United States
(12) Patent Application Publication
(10) Pub. No.: US 2026/0256958 A1
(43) Pub. Date: Sep. 3, 2026

(54) ZWITTERIONIC METAL CHELATORS

(71) Applicant: Curadel Surgical Innovations, Inc., Binita Springs, FL (US)

(72) Inventors: John V. Frangioni, Bonita Springs, FL (US); Wolfgang Maison, Winsen (DE)

(73) Assignee: Curadel Surgical Innovations, Inc., Bonita Springs, FL (US)

(21) Appl. No.: 19/558,087

(22) Filed: Mar. 5, 2026

Related U.S. Application Data
(63) Continuation-in-part of application No. 18/732,982, filed on Jun. 4, 2024.
Application No. 18/732,982 is a continuation-in-part of application No. 18/640,169, filed on Apr. 19, 2024, now Pat. No. 12,440,585.
(63) Continuation-in-part of application No. PCT/US2024/045968, filed on Sep. 10, 2024.
(60) Provisional application No. 63/538,038, filed on Sep. 12, 2023.
(60) Provisional application No. 63/767,817, filed on Mar. 6, 2025.

(51) Int. Cl. A61K 51/04; A61K 51/08

(52) U.S. Cl. CPC A61K 51/0482; A61K 51/082; A61K 51/083; A61K 51/088; A61K 2121/00; A61K 2123/00

(57) ABSTRACT

The present invention relates to zwitterionic metal chelators and their use as imaging, diagnostic, chemical processing, and treatment agents. These zwitterionic metal chelators have desirable properties that maximize solubility in aqueous environments, minimize non-specific interactions, and retain the ability to target thus resulting in an improved performance in a variety of medical, agricultural, and chemical processes. In in vivo and medical applications, zwitterionic metal chelators improve the signal-to-background ratio and therapeutic window as compared to other metal chelators while retaining high stability.

### CROSS-REFERENCE TO RELATED APPLICATIONS

This Application is a Continuation-in Part of PCT International Application Serial No. PCT/US2024/045968, filed on Sep. 10, 2024. PCT International Application Serial No. PCT/US2024/045968 application claims priority to U.S. Provisional Patent Application Ser. No. 63/538,038 filed Sep. 12, 2023, U.S. Utility patent application Ser. No. 18/640,169 filed Apr. 19, 2024, and U.S. Utility patent application Ser. No. 18/732,982 filed Jun. 4, 2024. This application further claims priority to U.S. Provisional Patent Application Ser. No. 63/767,817, filed Mar. 6, 2025. The disclosures of each of which are incorporated herein by reference in their entirety.

### FIELD OF THE INVENTION

The present invention relates to certain zwitterionic metal chelators and their use as imaging, diagnostic, chemical processing, and treatment agents. The zwitterionic metal chelators described herein, have desirable properties that maximize solubility in aqueous environments, minimize non-specific interactions, and retain the ability to target thus resulting in an improved performance in a variety of medical, agricultural, and chemical processes. In in vivo and medical applications, zwitterionic metal chelators improve the signal-to-background ratio and therapeutic window as compared to other metal chelators while retaining high stability.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Mar. 6, 2025, is entitled “1515138_105US42_SL.xml”, and is 6,235 bytes in size.

### BACKGROUND OF THE INVENTION

Cationic metals are insoluble in water. For this reason, whenever they are needed in a medical, agricultural, or chemical process they must be bound to, e.g. by coordination or chelation, to an organic compound that renders the complex soluble in aqueous environments and less toxic. Previously described metal chelators pay little attention to the polyionicity and sphere of hydration that is required to fully isolate the metal and thus result in unwanted non-specific interactions.

Chelated metals are commonly used in a variety of current imaging techniques. These techniques, like magnetic resonance imaging (MRI), single-photon emission computed tomography (SPECT), or positron emission tomography (PET), permit the detection of human diseases and conditions.

However, in the applications mentioned above, the metal isotope needs to be bound to metal chelators to prevent toxic effects and, for targeted agents, to maintain association with the targeting vector. Many common metal chelators, such as DOTA, PyC3A, and macropa, lack the ability to be tuned with respect to the pharmacokinetics, solubility, non-specific tissue/organ uptake, and plasma-protein binding. In addition, common metal chelators are not easily cleared from the body resulting in accumulation in off-target tissues and organs. For diagnostic imaging, this results in higher background. For radiotherapy, this results in toxicity without benefit.

Similarly, in any agricultural or chemical processes that require metal chelation, little attention is currently paid to the level of aqueous solubility attained or mechanisms to minimize non-specific interactions that either lower the yield of the process or fail to block side reactions.

Some research has been undertaken to provide derivatives of these chelators by allowing for the conjugation of targeting vectors, such as antibodies, peptides, small molecules, and steroids. These derivatives are often produced by replacement of one or more carboxylic acid arms of the chelators to include a targeting vector. However, such replacement often has a substantial impact on the chelating properties of the chelator—rendering their metal-binding properties inferior, if not altogether useless for biomedical applications. In some instances, replacement alters the biodistribution and/or clearance of the molecule, which can lead to a higher background or a smaller therapeutic window.

As such, there remains a need for new and improved agents with high stability that maximize the solubility of metal complexes in aqueous environments, minimize non-specific/off-target interactions, equilibrate rapidly between the intravascular and extravascular spaces when injected into the body and are then cleared efficiently from the body, including by renal filtration. The zwitterionic metal chelators of the invention are directed toward these and other needs.

### SUMMARY OF THE INVENTION

This invention provides for zwitterionic metal chelators which are useful for various medical, agriculture, and chemical processes. These chelators provide improved properties such as high solubility in aqueous environments and low non-specific interactions. When used in medical applications they can increase the signal-to-background ratio of imaged tissues and the therapeutic window of treated tissue, while allowing for easier and more efficient clearance by the subject.

In one aspect, the disclosure provides a zwitterionic metal chelator complex comprising a metal chelator having one or more zwitterionic groups and a metal or metal isotope selected from the group consisting of a radionuclide, a label, a paramagnetic metal and a heavy metal.

In certain embodiments, the metal chelator is a derivative of DOTP, macropaP, macropaN, or PYT. In still other embodiments, the zwitterionic metal chelator complexes further comprise one or more targeting vectors. In particular embodiments, the one or more targeting vectors are cRGD, PSMA-617, FAPI, octreotide, bombesin, or a homo- or hetero-dimer formed from their combination.

In some embodiments, wherein the metal chelator is a derivative of DOTP, the metal or metal isotope is Pb, Pd, Zr, Cu, Ga, In, Y, Gd, Lu, Ac, or Tb.

In other embodiments, wherein the metal chelator is DOTP, the metal or metal isotope is Pb.

In some embodiments, the zwitterionic metal chelator of the zwitterionic metal chelator complex has the formula:

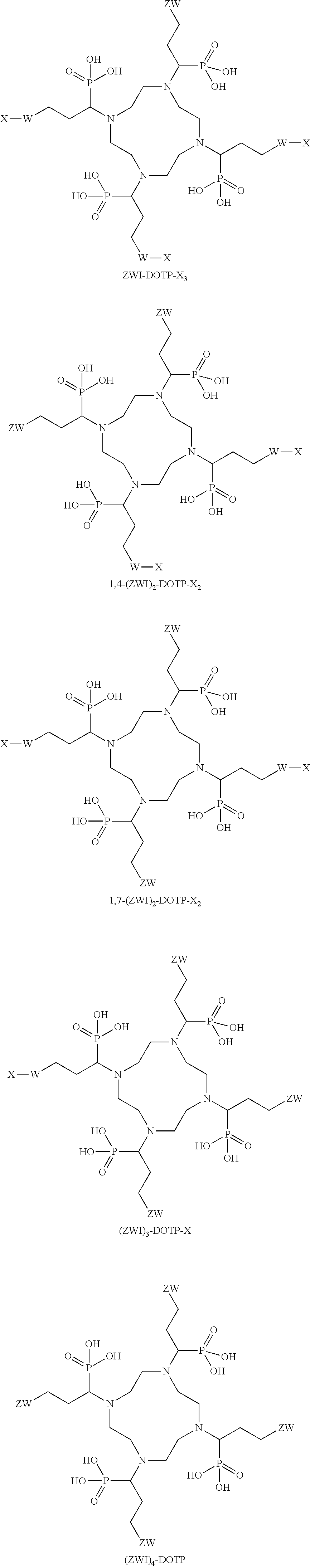

wherein ZW represents a zwitterionic group; each instance of W and Y independently represent a linking group, and each instance of X represents a reactive group. Substituents R are hydrogen or alkyl groups or a combination of both. In certain embodiments, ZW represents an ammonium sulfobetaine group. In certain embodiments, ZW represents an N-oxide group. In certain embodiments, X may also include a targeting vector.

In certain embodiments, the metal chelator is a derivative of macropaP or macropaN and the metal or metal isotope is Pd, Pb, Zr, Cu, Ga, In, Y, Gd, Lu, Ac or Tb.

In some embodiments, in the metal chelator is a derivative of macropaP or macropaN and wherein the zwitterionic metal chelator of the zwitterionic metal chelator complex has the formula:

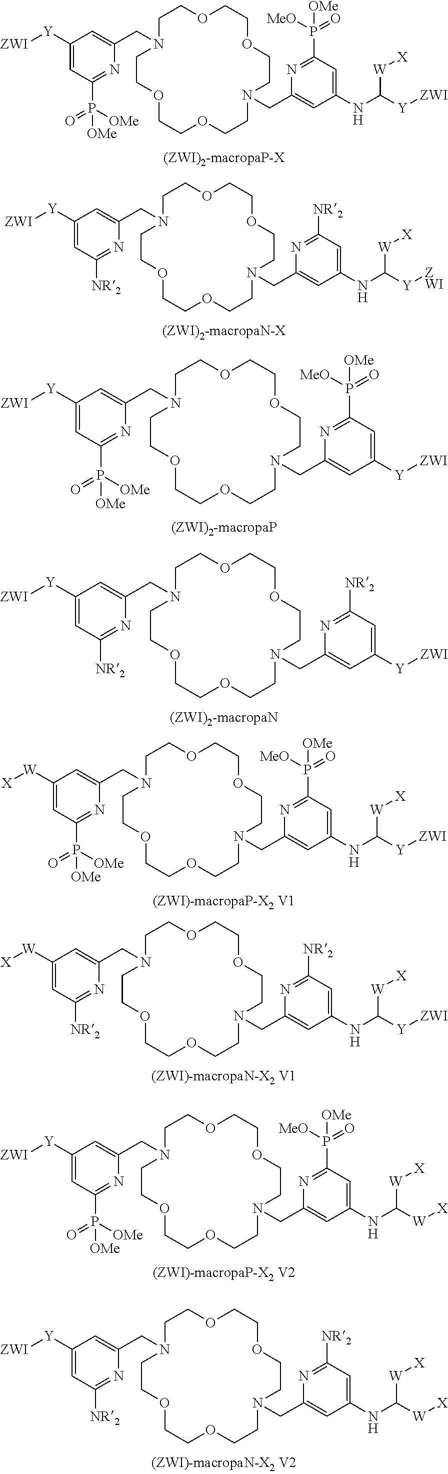

wherein ZWI represents a zwitterionic group; each instance of W and Y independently represent a linking group, and each instance of X represents a reactive group and/or a targeting vector.

In certain embodiments, the metal chelator is a derivative of PYT and the metal or metal isotope is Pd, Pb, Zr, Cu, Ga, In, Y, Gd, Lu, Ac or Tb.

In some embodiments, the metal chelator is a derivative of PYT and wherein the zwitterionic metal chelator of the zwitterionic metal chelator complex has the formula:

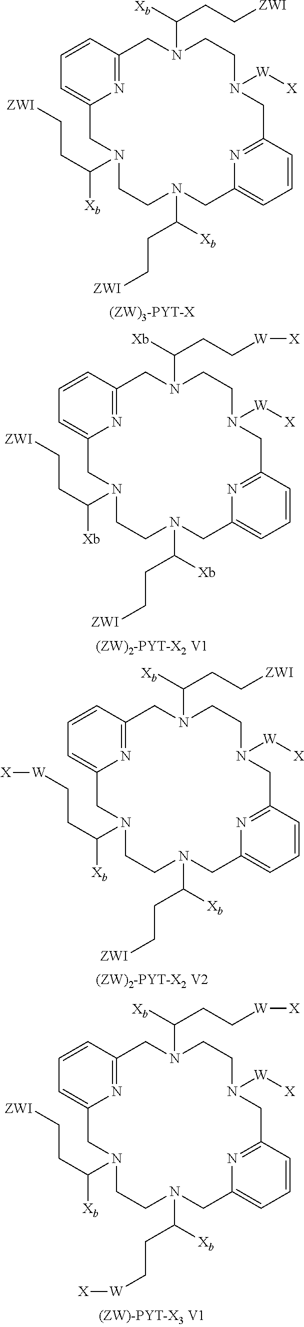

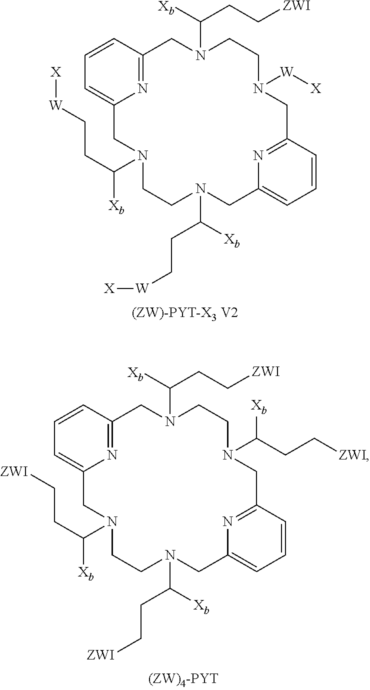

wherein ZWI represents a zwitterionic group; each instance of W and Y independently represent a linking group, each instance of X_{b} may be —COOH, —PO(OH)₂, or —NR′₂; each instance of R′ is independently hydrogen or an alkyl group; and each instance of X represents a reactive group and/or a targeting vector.

In certain embodiments, the zwitterionic metal chelator complexes further comprise one or more targeting vectors. In particular embodiments, the one or more targeting vectors are cRGD, PSMA-617, FAPI, octreotide, a bombesin analog, or a homo- or hetero-dimer formed from their combination.

In particular, these chelators allow for lower temperature, and even ambient temperature, chelation of metals. This fast, room temperature chelation allows for more efficient attachment to proteins including, but not limited to monoclonal antibodies which is not achievable with previously known chelators, such as DOTA.

In another aspect, the disclosure provides an imaging agent comprising a zwitterionic metal chelator complex according to the disclosure.

In another aspect, the disclosure provides a method of imaging cells, tissues, or organs, the method comprising:
(a) contacting cells with an imaging agent according to the disclosure; and
(b) imaging the cells, tissues, or organs using positron emission tomography (PET), single-photon emission computerized tomography (SPECT), or magnetic resonance imaging (MRI).

In some embodiments of the method of imaging, the cells are tumor cells or cells undergoing angiogenesis.

In other embodiments of the method of imaging, t the imaging agent is administered to an organism comprising or suspected of comprising the cells.

In particular embodiments of the method of imaging, the organism is human.

In some embodiments of the method of imaging, the tissue or cells is imaged in vivo.

In another aspect, the disclosure provides therapeutic agent comprising a zwitterionic metal chelator complex according to the disclosure and a pharmaceutically acceptable carrier or excipient.

In another aspect, the disclosure provides a method of treating a cancerous condition in a subject in need thereof, the method comprising:
contacting cancer cells in the subject with an effective amount of a therapeutic agent according to the disclosure,
wherein the metal atom complexed to the zwitterionic metal chelator is:
   a radioactive metal isotope known to emit ionizing radiation that results in the death of cells that take up the analogs;
   or a non-radioactive metal that is capable of releasing cytotoxic radiation upon irradiation with alpha emission, beta emission, neutron capture, or a combination thereof.

In some embodiments of the method of treatment, the metal atom complexed to the zwitterionic metal chelator is a non-radioactive metal that is capable of releasing cytotoxic radiation upon irradiation with alpha emission, beta emission, neutron capture, or a combination thereof; the method further comprising a step of irradiating the tumor cells using alpha emission, beta emission, neutron capture, or a combination thereof.

In some other embodiments of the method of treatment, the cancer cells are adult solid tumor cells or pediatric solid tumor cells.

In other embodiments of the method of treatment, the cancer cells are melanoma cells, neuroblastoma cells, lung cancer cells, adrenal cancer cells, colon cancer cells, colorectal cancer cells, ovarian cancer cells, prostate cancer cells, liver cancer cells, subcutaneous cancer cells, squamous cell cancer cells, intestinal cancer cells, retinoblastoma cells, cervical cancer cells, glioma cells, breast cancer cells, pancreatic cancer cells, Ewings sarcoma cells, rhabdomyosarcoma cells, osteosarcoma cells, retinoblastoma cells, Wilms' tumor cells, and pediatric brain tumor cells.

In some embodiments of the method of treatment, the cancer cells are prostate cancer cells. In some other embodiments of the method of treatment, the cancer cells are malignant cancer cells.

In another aspect, the disclosure provides method of treating a non-cancerous condition in a subject in need thereof, the method comprising:
administering to the subject an effective amount of a therapeutic agent according to the disclosure,
wherein the metal atom complexed to the zwitterionic metal chelator is:
   a radioactive metal isotope known to emit ionizing radiation that results in a therapeutic effect on the subject.
   or a non-radioactive metal that is capable of releasing therapeutic radiation upon irradiation with alpha emission, beta emission, neutron capture, or a combination thereof.

In some embodiments of the method of treatment, the non-cancerous condition is a musculoskeletal disorders or a tissue hypertrophy disorder.

In some embodiments of the method of treatment, the subject is a human.

In another aspect, the disclosure provides a diagnostic agent comprising a zwitterionic metal chelator complex according to the disclosure and a pharmaceutically acceptable carrier or excipient.

In another aspect, the disclosure provides a method of measuring the efficacy of a biological system of a subject, the method comprising:
(a) administering a quantifiable amount of a diagnostic agent according to the disclosure to the subject;
(b) imaging the subject with positron emission tomography (PET), single-photon emission computerized tomography (SPECT), or magnetic resonance imaging (MRI); and
(c) determining the amount of therapeutic agent present in the biological system being observed in the subject.

In some embodiments of the method of measuring the efficacy of a biological system, the biological system is the renal system, the hepatic system, or the blood pool.

In another aspect, the disclosure provides a method of measuring the efficacy of renal function a subject, the method comprising:
(a) administering a quantifiable amount of a diagnostic agent according to the disclosure to the subject;
(b) imaging the subject with positron emission tomography (PET), single-photon emission computerized tomography (SPECT), or magnetic resonance imaging (MRI); and
(c) determining the amount of therapeutic agent present in the biological system being observed in the subject.

In another aspect, the disclosure provides a method of quantifying the glomerual filtration rate of a subject, the method comprising:
(a) administering a quantifiable amount of a diagnostic agent according to the disclosure to the subject;
(b) determining the amount of diagnostic agent present in the blood and urine of the subject as a function of time using either measurements of each bodily fluid or imaging the subject with positron emission tomography (PET), single-photon emission computerized tomography (SPECT), or magnetic resonance imaging (MRI)

In another aspect, the disclosure provides method for removing toxic or excess metals in a subject in need of such treatment, the method comprising:
(a) administering a therapeutically effective amount of a therapeutic agent according to the disclosure to the subject,
wherein the therapeutic agent comprises a metal chelator having one or more zwitterionic groups which is not coordinated to a metal or metal isotope, and a pharmaceutically acceptable carrier or excipient.

In other embodiments of the method of removing toxic or excess metals in a subject, the toxic or excess metals are gadolinium.

In another aspect, the disclosure provides a radiosurgical method for treating a patient body, the method comprising:
receiving a desired lesion pattern and planned radiation distribution;
   administering an effective amount of a diagnostic agent according to the disclosure to the subject to effectively image the desired lesion pattern;
performing surgery on the desired lesion pattern to treat the patient body.

In some embodiments of the radiosurgical method of the disclosure, the zwitterionic metal chelator of the diagnostic agent further comprises one or more targeting vectors wherein the one or more targeting vectors are cRGD, PSMA-617, FAPI, octreotide, a bombesin analog, or a homo- or hetero-dimer formed from their combination.

In other embodiments of the radiosurgical method of the disclosure, the desired lesion pattern is received from a user interface of a treatment planning module.

In still other embodiments of the radiosurgical method of the disclosure, the treatment planning module is pre-programmed with specifications for various disease states and cancerous conditions.

In yet other embodiments of the radiosurgical method of the disclosure, the the treatment planning module identifies lesion patterns for various disease states and cancerous conditions using artificial intelligence data.

In particular embodiments of the radiosurgical method of the disclosure, the n the surgery is performed using a sterotactic radiosurgical system.

In another aspect, the disclosure provides a method of treating a cancer by administering an effective amount of a therapeutic agent according to the disclosure, wherein the method comprises a step to diagnose the cancer and a step of administering the therapeutic agent to a subject determined to be in need thereof;
wherein the step to diagnose the cancer comprises:
contacting cells, tissues or organs of a subject with an imaging agent,
imaging the cells, tissues, or organs of the subject using positron emission tomography (PET), single-photon emission computerized tomography (SPECT), or magnetic resonance imaging (MRI), and
diagnosing the cancer in the cells tissues, or organs of the subject based on imaging data collected;
and wherein the metal atom complexed to the zwitterionic metal chelator is:
   a radioactive metal isotope known to emit ionizing radiation that results in the death of cells that take up the analogs;
   or a non-radioactive metal that is capable of releasing cytotoxic radiation upon irradiation with alpha emission, beta emission, neutron capture, or a combination thereof.

### BRIEF DESCRIPTION OF THE DRAWINGS

FIG. 1a is a representation of five DOTP-based zwitterionic metal chelators according to the invention.

FIG. 1b is a representation of four macropaP-based zwitterionic metal chelators and four macropaN-based zwitterionic metal chelators according to the invention.

FIG. 1c is a representation of six PYT-based zwitterionic metal chelators according to the invention.

FIG. 2 is a representation of Zwitterionic groups according to the invention.

FIG. 3 is a representation of three targeting vectors encompassed by the invention.

FIG. 4 depicts a solid-phase synthetic scheme (Scheme 3) which produces an azide functionality to which the zwitterionic groups can be added using click-chemistry methods as described herein and as would be familiar to one of the art.

FIG. 5 is a representation of seven non-targeted zwitterionic metal chelators according to the invention.

FIG. 6 is a representation of seven targeted zwitterionic metal chelators according to the invention (targeting vector=tv).

FIG. 7 is a representation of Zwitterionic groups according to the invention.

FIG. 8 is a representation of three targeting vectors encompassed by the invention.

FIG. 9 is a representation of the synthesis of DOTA-based zwitterionic conjugates.

FIG. 10 is a representation of the synthesis of deferoxamine-based zwitterionic conjugates.

### DETAILED DESCRIPTION

The present disclosure relates, inter alia, to an imaging, diagnostic, therapeutic or chemical agent that is composed of a zwitterionic metal chelator. In some aspects, the agents described herein are useful in, for example, the detection of abnormal or diseased biological tissues and cells. In some aspects, the zwitterionic metal chelators are particularly useful for imaging whole organisms as they have improved in vivo behavior, such as low non-specific binding to non-targeted tissues and high stability, resulting in an improved signal-to-background ratio in connection with the detected signal and/or improved therapeutic window. Similarly, in an agricultural or chemical process that now uses a metal chelator, zwitterionic metal chelators will improve solubility and minimize non-specific interactions. It is believed that these improved properties result from the balancing of formal charges on the metal chelator, rendering a polyionic yet “charge-balanced” molecule having a net charge that is neutral or close to neutral, with an extended sphere of hydration and better isolation of the metal.

### Definitions and Additional Embodiments

The following definitions will be useful in understanding the instant invention.

As used herein, the term “comprising” is intended to mean that the compositions and methods include the recited elements, but do not exclude other elements. “Consisting essentially of”, when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

As used in the specification and claims, the singular form “a”, “an” and “the” include plural references unless the context clearly dictates otherwise.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless specifically stated or obvious from context, as used herein, the term “or” is understood to be inclusive.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

As used herein, the term “subject” or “patient” encompasses mammals and non-mammals. Examples of mammals include, but are not limited to, humans, chimpanzees, apes monkeys, cattle, horses, sheep, goats, swine; rabbits, dogs, cats, rats, mice, guinea pigs, and the like. Examples of non-mammals include, but are not limited to, birds, fish, parasites, microbes, and the like.

As used herein, the term “administration” or “administering” of the subject compound refers to providing a zwitterionic metal chelator of the invention and/or prodrugs thereof to a subject in need of diagnosis or treatment. As used herein, the term “ligand”, “targeting vector”, or “targeting ligand” refers to a moiety which is bound to or coordinated to the therapeutic/imaging agents or zwitterionic metal chelators of the composition of the invention to provide enhanced binding to particular cell types or an increased concentration in the presence of particular cell types. In certain embodiments, the targeting vector can be bound to the therapeutic/imaging agents or zwitterionic metal chelators of the compositions in addition to the zwitterionic groups thereon. In still other embodiments, the targeting vector can be bound to the zwitterionic metal chelator in place of one or more zwitterionic groups provided that the zwitterionic metal chelator retains at least one zwitterionic group.

As used herein, the term “therapeutic window” or “therapeutic index” refers to the relationship between the therapeutic and toxic dose of a given drug and is calculated using the ED50 and TD50 (Therapeutic Index=TD50/ED50). In certain embodiments of the invention, the zwitterionic metal chelators of the invention have a higher therapeutic index relative to other metal chelators. In certain other embodiments, the therapeutic window refers to a certainty safety factor (CSF) which is defined herein as the ratio of [TD₁/ED₉₉]. A CSF>1 indicates that the dose effective in 99% of the population is less than the dose that would be toxic in 1% of the population. In certain embodiments of the invention, the zwitterionic metal chelators of the invention have a higher CSF relative to other metal chelators.

As used herein, the term “carrier” refers to chemical compounds or agents that facilitate the incorporation of a compound described herein into cells or tissues.

As used herein, the term “acceptable” with respect to a formulation, composition or ingredient, as used herein, means having no persistent detrimental effect on the general health of the subject being treated.

As used herein, the term “diluent” refers to chemical compounds that are used to dilute a compound described herein prior to delivery. Diluents can also be used to stabilize compounds described herein.

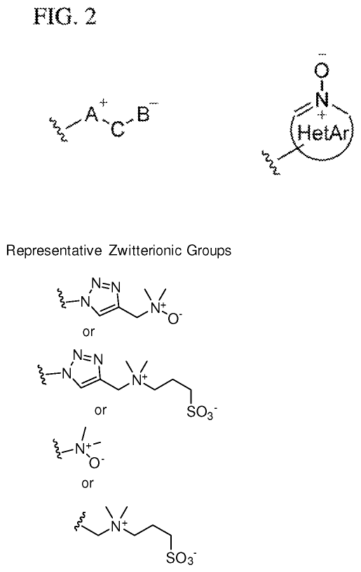

As used herein, the term “zwitterionic group,” “zwitterionic ligand,” or “zwitterion” refer to one or more charged moieties or ligands which are present on or can be reacted with a metal chelator core. The zwitterionic metal chelators of the claimed invention are decorated by one or more zwitterionic groups, i.e., moieties combining positive (A+, FIG. 2) and negative charges (B−, FIG. 2) that sum to a net charge of zero. These zwitterionic groups are distinct from the chelator core itself, which often has negative charges to chelate positively-charged metals. For example, a zwitterionic metal chelator with a chelator core of −4 that binds a +4 metal would have a total net charge of zero. Although a total net charge of zero is considered ideal, a zwitterionic metal chelator with a chelator core of −4 that binds a +2 metal, resulting in an overall charge of −2, would still be expected to exhibit improved properties in vivo because of shielding of the chelator core/metal complex by one or more zwitterionic groups. In the absence of zwitterionic groups, the molecule would have no such shielding or expanded water of hydration and would be more likely to bind non-specifically.

A particular active agent molecule may have several attached “zwitterionic groups” or charge pairs (also described herein as “ZW group(s)”). In general, the anion portion and the cation portion of the zwitterionic group (charge pair) will be part of the same moiety, though it is possible for two ionic groups to be used as separate moieties to form a zwitterionic group. In particular embodiments, the zwitterionic group is covalently bound to the base structure via a carbon-carbon bond, a carbon-oxygen bond, or a nitrogen-carbon bond. Examples of zwitterionic groups (charge pairs) that can be included in the compounds and complexes of the claimed invention include, but are not limited to, ammoniophosphates, ammoniophosphonates, ammoniophosphinates, ammoniosulfonates, ammoniosulfates, ammoniocarboxylates, ammoniosulfonamides, ammonio-sulfon-imides, guanidiniocarboxylates, pyridiniocarboxylates, pyridiniosulfonates, ammonio(alkoxy)dicyanoethenolates, ammonioboronates, sulfoniocarboxylates, phophoniosulfonates, and phosphoniocarboxylates. The charged groups in these zwitterions can be separated by suitable spacer groups (C in FIG. 2) like linear or branched alkyl chains, aryl or heteroaryl moieties. In certain embodiments, the zwitterionic groups can be derivatives of amino acids, such as amino carboxylic acids, amino phosphonic acids, amino phosphinic acids or amino sulfonic acids, furthermore, aminoalkyl substituted sulfates or phosphates. Zwitterions can also be derivatives of betaines, such as carboxybetaines, sulfobetaines, sulfabetaines, phosphobetaines or phosphabetaines or N-oxides or derivatives of sulfamic acid. Particular examples of zwitterionic groups include ammonium sulfobetaines or N-oxides. A simple example of a zwitterionic group at physiological pH is the charge pair of a carboxylic acid (deprotonated at physiological pH) and an amine (protonated at physiological pH).

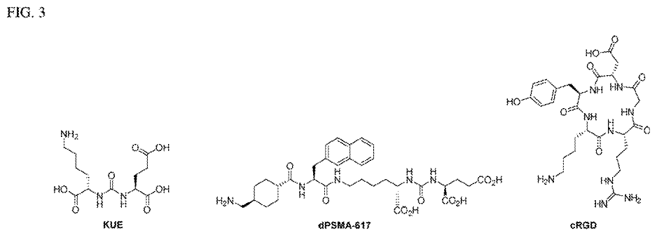

In some embodiments, the zwitterionic metal chelators of the invention can also comprise a targeting vector for an agricultural process, chemical process, disease, or tissue-specific epitope, such as the cyclic peptide cRGDyK (aka cRGD, FIG. 3) bound to one or more arms of the metal chelator. cRGD is a cyclic derivative of the tripeptide Arg-Gly-Asp which can be conjugated to one or more arms of the metal chelators of the invention. In still other embodiments, the targeting vector is octreotide or a bombesin analog. In other embodiments, the targeting vector is KUE or dPSMA-617, a GPI-derivative, a small molecule capable of targeting Fibroblast Activation Protein (FAP) also called FAP-inhibitor or FAPI, an amino acid or combination of amino acids, or derivatives thereof. In such embodiments, the targeting vector-conjugates can be formed in place of one or more zwitterionic groups. In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC(SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin.

An ideal zwitterionic metal chelator conjugated to a targeting vector would adopt the total net charge of the targeting vector, which is purposeful because in most cases the charges on the targeting vector are crucial for the ability to bind its target. Targeted zwitterionic metal chelators thus retain the major advantage of minimizing non-specific binding while maximizing specific binding. It should be apparent to those skilled in the art that additional charges can be added to the zwitterionic metal chelator, if needed, to balance overall surface charge to zero.

In certain embodiments, the zwitterionic metal chelators of the invention chelator comprise a reactive linking group. Such reactive linking groups are typically an activated derivative of a carboxylic acid, such as an N-hydroxysuccinimide (NHS) ester, a sulfo-NHS ester, a pentafluorophenyl (PFP) ester, a hydroxybenzotriazole (HOBt) ester, a hydroxyazabenzotriazole (HOAt) ester, a tetrafluorophenyl (TFP) ester, an acid anhydride, an acid azide or an acid halide. Such reactive linking groups can be bound or substituted onto the chelator at any suitable structural location as would be understood by one of ordinary skill in the synthesis of such compounds. Reactive linking groups also include, but are not limited to, alkynes, azides, maleimides, thiols, amines, alkohols, phenols, carbonyls, phosphanes, alkenes and tetrazines.

As used herein, the term “contacting” refers to the bringing together of substances in physical contact such that the substances can interact with each other. For example, when an agent is “contacted” with tissue or cells, the tissue or cells can interact with the agent, for example, allowing the possibility of binding interactions between the agent and molecular components of the tissue or cells. “Contacting” is meant to include the administration of a substance such as an agent of the invention to an organism. Administration can be, for example, oral or parenteral.

As used herein, the term “ionic group” refers to a moiety comprising one or more charged substituents. The “charged substituent” is a functional group that is generally anionic or cationic when in substantially neutral aqueous conditions (e.g. a pH of about 6.5 to 8.0 or about physiological pH (7.4)). As recited above, examples of charged anionic substituents include anions of inorganic and organic acids such as sulfonate (—SO₃¹⁻), oxide, sulfinate, carboxylate, phosphinate, phosphonate, phosphate, and esters (such as alkyl esters) thereof. In some embodiments, the charged substituent is sulfonate or oxide. Examples of charged cationic substituents include quaternary ammonium ions (—NR₃⁺) and phosphonium ions (—PR₃⁺), where R is independently selected from C₁₋₆ linear alkyl, C₄₋₆ branched alkyl, C₃₋₆ cycloalkyl, aryl, heteroaryl and arylalkyl or heteroarylalkyl. Other charged cationic substituents include protonated primary, secondary, and tertiary amines, as well as guanidinium or amidinium or pyridinium or other protonated, alkylated or oxygenated nitrogen heterocycles. In some embodiments, the charged substituent is —N(CH₃)₃⁺.

As used herein, the phrase “non-ionic oligomeric or polymeric solubilizing groups” refers to soluble polymers such as, for example, polyethylene glycol, polypropylene glycol, polyethylene oxide and propylene oxide copolymer, a carbohydrate, a dextran, polyacrylamide, a peptide and the like. The solubilizing group can be attached by any desired mode. The point of attachment can be, e.g., a carbon-carbon bond, a carbon-oxygen bond, or a nitrogen-carbon bond. The attachment group can be, e.g., an ester group, a carbonate group, an urea group, an alcohol group, an ether group, a sulfide group, an amino group, an alkylene group, an alkyne group, an azide group, a tetrazine, an amide group, a carbonyl group, or a phosphate group.

Some examples of solubilizing groups include polyethylene glycols, such as —(CH₂CH₂O)ₐ—H, —OC(═O)O(CH₂CH₂O)ₐH, —OC(═O)O(CH₂CH₂O)ₐCH₃, —O(CH₂CH₂O)ₐCH₃, and —S(CH₂CH₂O)₂CH₃, “a” being an integer between about 2 and about 250. In some embodiments, “a” is 4 to 12 or 5 to 10. In further embodiments, “a” is 6, 7, or 8. Other examples of solubilizing groups include dextrans such as —OC(═O)O(dextran).

The solubilizing moiety can have an absolute molecular weight of from about 500 amu to about 100,000 amu, e.g., from about 1,000 amu to about 50,000 amu or from about 1,500 to about 25,000 amu.

Further examples of solubilizing groups include: —(CH₂)_{c}—(OCH₂CH₂)_{d}—OR^{a}, wherein “c” is 0 to 6, “d” is 1 to 200, and R^{a} is H or C₁₋₆ alkyl. In some embodiments, “c” is 1 to 4, “d” is 1 to 10, and R^{a} is H. In some embodiments, “d” is 6 or 7.

See WO 2008/017074, U.S. Ser. No. 12/376,243 (filed Feb. 3, 2009), and U.S. Ser. No. 12/376,225 (filed Feb. 3, 2009), each of which is incorporated herein by reference in its entirety, for a further description of suitable non-ionic oligomeric or polymeric solubilizing groups, and method for incorporating them into dyes.

As used herein, the term “azido derivative(s)” refers to chemical compounds that contain one or more azido groups (—N₃) attached to their molecular structure. In certain embodiments, the azido group is a functional group consisting of three nitrogen atoms (N) connected in a linear arrangement, typically represented as —N₃. In certain embodiments, the azido derivatives include one or more of Fmoc-Azidoalanine, Fmoc-Azidohomoalanine, Fmoc-Azidophenylalanine, Fmoc-Azidoglycine, Fmoc-Azidoornithine, Fmoc-Azidolysine, Fmoc-Azidoaspartic Acid.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Compounds of the invention can also include all isotopes of atoms occurring in the intermediates or final compounds. Isotopes include those atoms having the same atomic number but different mass numbers. For example, isotopes of hydrogen include tritium and deuterium.

The chemical substances represented herein by name, chemical formula, or structure are meant to include all stereoisomers, geometric isomers, tautomers, resonance structures, and isotopes of the same, unless otherwise specified.

The chemical substances described herein may be charged or include substituents with formal charges. When such chemical substances are represented as charged, it is understood that, unless otherwise specified, the charges are generally countered with an appropriate counterion. For example, chemical substances or functional groups having a charge of −1 are understood to be countered with an ion have a +1 charge. Suitable counterions with +1 charge include Na+, K+, tetraalkylammonium ions, and the like. Conversely chemical substances or functional groups having a charge of +1 are understood to be countered with an ion having a −1 charge. Suitable counterions with −1 charge include F—, Cl—, Br—, I—, sulfate, phosphate, perchlorate, acetate, trifluoroacetate, maleate, fumarate, mesylate, lactate, pyruvate, laevulinate, gluconate and the like.

Zwitterionic Metal Chelators Coordinated to a Metal Isotope In certain aspects, the disclosure is directed to zwitterionic metal chelators coordinated to or labeled with a metal or metal isotope. In certain embodiments, the zwitterionic metal chelators coordinated to or labeled with a metal or metal isotope can be used medical, agricultural, or chemical processing applications.

Exemplary medical applications include applications for detection, imaging, or treatment in/of a subject or in a biological sample In certain embodiments, the disclosure further provides for treatment of benign and malignant tumors and tumor cells using a zwitterionic metal chelators labeled with a radioactive metal isotope known to emit ionizing radiation in a form that would result in the death of cells that take up the analogs labeled with the radioactive metal isotope. Metal chelators labeled with certain non-radioactive metal isotopes, such as Zr or Gd, can be used for neutron capture therapy.

Various zwitterionic metal chelators of the invention, particularly chelators suitable for use in the imaging methods provided by the invention, include one or more metals or radioisotopes capable of emitting one or more forms of radiation or other contrast (for example, effects on water relaxation) which are suitable for detection with any standard radiology method such as PET, SPECT, gamma cameras, MRI and the like.

For the disclosed methods of detecting/imaging benign or malignant tissues, any metal isotope known to emit radiation in a form that is readily detectable by conventional imaging means can be incorporated into the targeting backbone. Non-limiting examples of “conventional imaging means” include gamma ray detection, PET scanning, SPECT scanning, and MRT scanning. Non-limiting examples of metals that may be complexed with the DOTP derivative chelators of the invention include Gd, Mn, Cu, Co, Y, In, Ga, Zr, Tc, Eu, Tb, Ac, Lu and other lanthanide or actinide metals. In some embodiments, the metals can be radioactive metal isotopes. Non-limiting examples of radioactive metal isotopes that may be used include Ga-66, Ga-67, Ga-68, Cu-64, Cu-67, Y-86, Co-55, Zr-89, Sr-83, Mn-52, As-72, Sc-44, Gd-153, Co-57, In-111, Ac-225, Tb-152, Tb-155 or Tc-99m.

For the disclosed methods of therapeutically treating malignant tumors, any radioactive metal isotope known to emit ionizing radiation in a form that would result in the death of cells that take up the analogs labeled with the radioactive metal isotope can be incorporated by chelation in the zwitterionic metal chelators of the invention. So, too, can non-radioactive metals that are capable of capturing neutrons and releasing cytotoxic radiation (neutron capture therapy). In some embodiments, the radioactive metal isotope emits its ionizing radiation in a form that minimizes damage to tissue outside of the cells that take up the labeled analogs. In certain embodiments, the invention provides compounds comprising one or more radioisotope suitable for use in radiation therapy. In certain embodiments, the zwitterionic metal chelators of the invention comprise at least one radioactive isotope of technetium, rhenium, gallium, indium, copper, yttrium, actinium, bismuth, samarium, dysprosium, holmium, terbium, or lutetium, including radioactive isotopes selected from Tc-99m, Tc-94m, Re-186, Re-188, Ga-68, Cu-64, Cu-67, Y-90, Y-86, Ac-225, Bi-213, In-111, Sm-153, Ho-166, Lu-177, Sc-43, Sc-44, Sc-47, Tb-149, Tb-152, Tb-155, Tb-161, and Dy-166. In other embodiments, non-limiting examples of metals used in neutron capture therapy include Zr-88 or Gd-157.

For the disclosed methods, the oxidation state of the metal coordinated to the chelator is not particularly limited. In general, the oxidation state can be adjusted based on the particular zwitterionic metal chelator used and the particular medical application used. In certain embodiments, the metal coordinated to the chelator has an oxidation state ranging from +1 to +5.

Various zwitterionic metal chelators of the invention are capable of generating at least a 2:1 target or signal to background ratio of radiation intensity, or more preferably about a 5:1, about a 10:1 or about a 15:1 ratio of radiation intensity between target and background.

Further, the zwitterionic metal chelators of the invention are excreted from tissues of the body quickly, and without significant non-specific uptake by off-target tissues and organs, to prevent prolonged exposure to the radiation of a radiolabeled compound or the toxic effects of non-radioactive metals administered to the patient. Typically zwitterionic metal chelators of the invention are eliminated from the body in less than about 24 hours. More preferably, compounds of the invention are eliminated from the body in less than about 16 hours, 12 hours, 8 hours, 6 hours, 4 hours, 2 hours, 90 minutes, or 60 minutes. Typically preferred compounds are eliminated in between about 60 minutes and about 120 minutes.

In some embodiments, the clearance of the zwitterionic metal chelators comprising the imaging agent or therapeutic agent is from the tumor, tissue, or organ in the subject. In some embodiments, there is more rapid clearance of the zwitterionic metal chelators comprising the imaging agent from the kidneys than from the tumor of the subject.

In some embodiments, the zwitterionic metal chelators are stable in vivo such that substantially all, e.g., more than about 50%, 60%, 70%, 80%, or more preferably 90% of the injected compound is not metabolized by the body prior to excretion. In other embodiments, the zwitterionic metal chelators are stable in vivo.

### DOTP—Based Zwitterionic Metal Chelators

In one aspect, the invention provides a zwitterionic metal chelator based on 1,4,7,10-tetraazacyclododecane-1,4,7,10-(tetra(methylene phosphonic acid, also known as DOTP. DOTP is a derivative of DOTA in which —COOH groups are replaced with —PO(OH)₂ groups. Such zwitterionic metal chelators include the following:

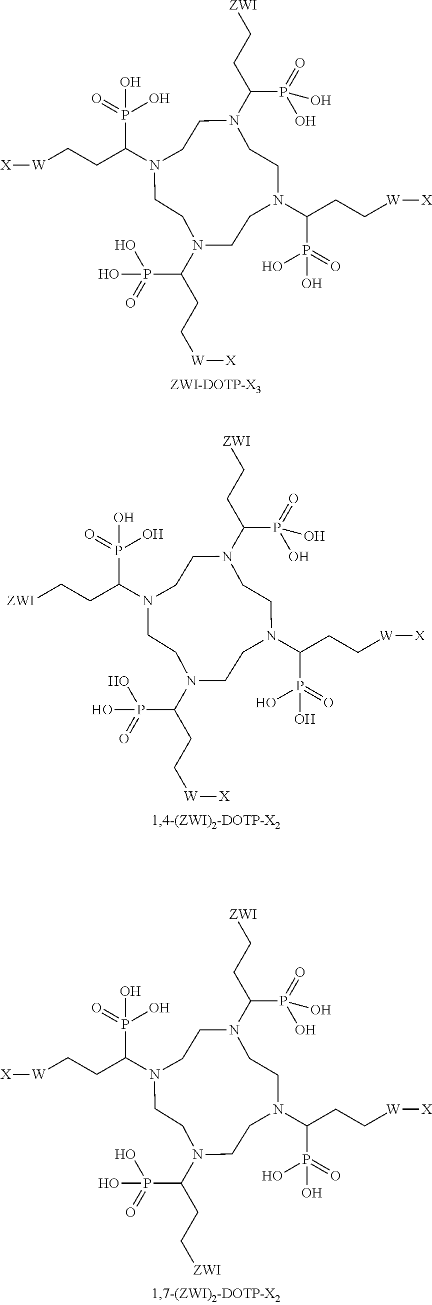

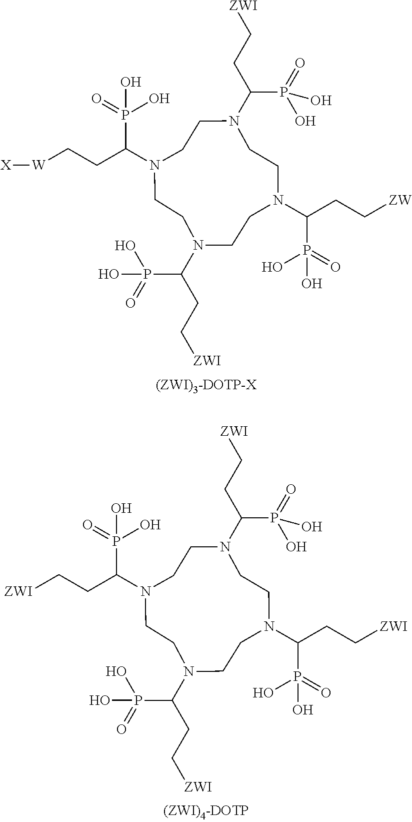

in which ZWI represents a zwitterionic group; each instance of W and Y independently represent a linking group, and each instance of X represents a reactive group and/or a targeting vector. In certain embodiments, ZWI represents an ammonium sulfobetaine group. In other embodiments ZWI represents an N-oxide group. In some embodiments, the zwitterionic group (ZWI) is optionally coordinated through click-chemistry (i.e., through a triazole group).

Representative ZWI groups include those shown in FIG. 2 and, for example, those shown below:

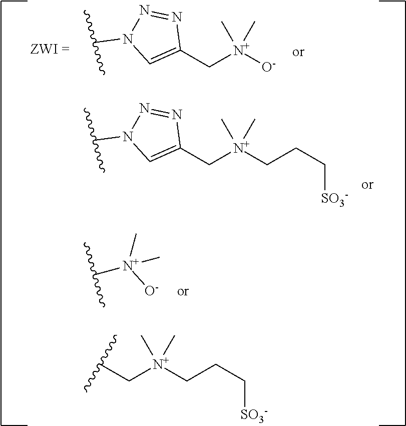

In certain embodiments, reactive groups X for conjugation to targeting vectors or other molecules or materials. Such reactive groups can be be a carboxylic acid, an activated derivative of a carboxylic acid, such as an NHS ester, a sulfo-NHS ester, a PFP ester, a HOBt ester, a HOAt ester, a TFP ester, an acid anhydride, an acid azide or an acid halide. The reactive group X can also be an amine, azide, alkyne, alkene, ketone, aldehyde, alcohol, phenol, maleimide, thiol, phosphane or a tetrazine. The zwitterionic groups ZWI and the reactive groups X can be separated from the chelator core structure by appropriate spacer moieties W and Y including alkyl, aryl or heteroaryl groups. In certain embodiments, each of W and Y may independently be absent.

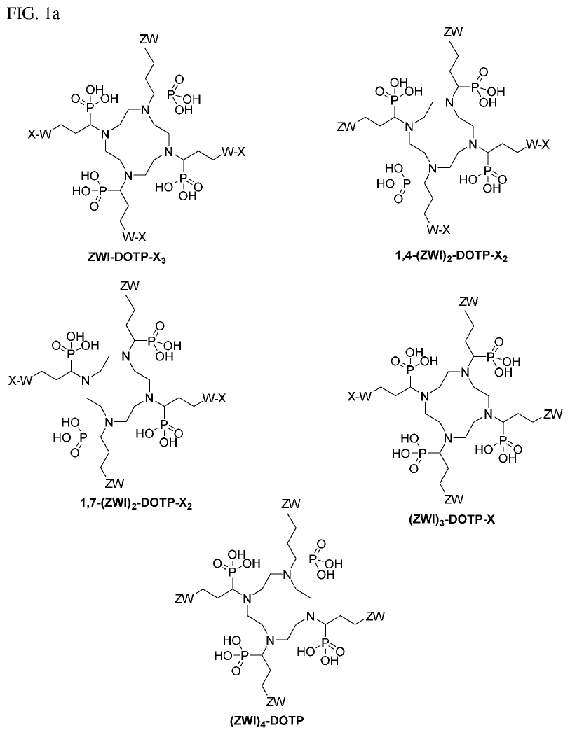

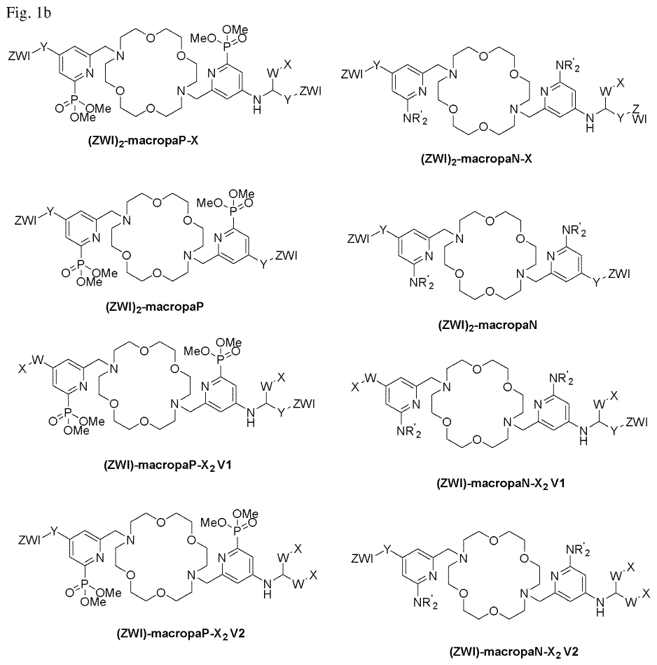

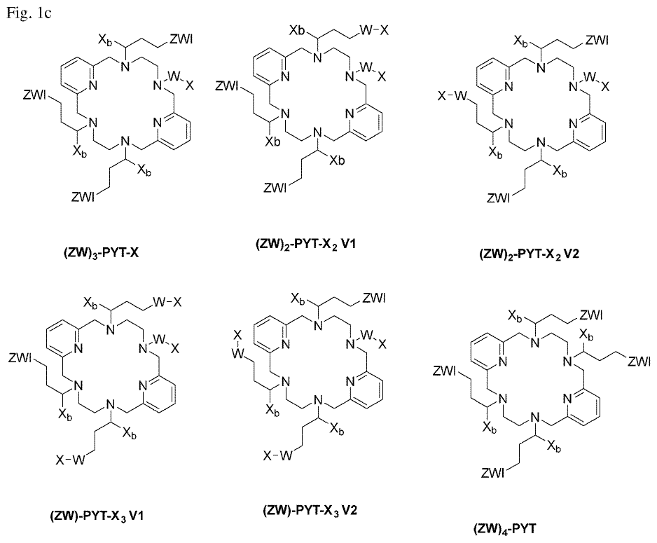

In certain embodiments, a DOTP-based zwitterionic metal chelator contains stereogenic centers, any of these might have R or S-configuration. The DOTP-based zwitterionic metal chelator might be a single stereoisomer or might be a mixture of stereoisomers. The DOTP-based zwitterionic metal chelator includes 1, 2, 3, or 4 zwitterionic groups as defined in FIG. 3 and 1, 2 or 3 reactive groups X for conjugation to targeting vectors or other molecules or materials. This reactive group can be a carboxylic acid, an activated derivative of a carboxylic acid, such as an NHS ester, a sulfo-NHS ester, a PFP ester, a HOBt ester, a HOAt ester, a TFP ester, an acid anhydride, an acid azide or an acid halide. The reactive group X can also be an amine, azide, alkyne, alkene, ketone, aldehyde, alcohol, phenol, maleimide, thiol, phosphane or a tetrazine. The zwitterionic groups ZWI and the reactive groups X can be separated from the chelator core structure by appropriate spacer moieties W and Y including alkyl, aryl or heteroaryl groups. In particular embodiments, the DOTP-based zwitterionic metal chelator includes 1, 2, 3, or 4 zwitterionic groups and 4 carboxylate groups for complexation to the metal or metal isotope. In still other embodiments, the DOTP-based zwitterionic metal chelator includes 1, 2, 3, or 4 zwitterionic groups, 4 carboxylate groups for complexation to the metal or metal isotope, and one or more targeting vectors. These targeting vectors can be bound to the chelator at any suitable structural location (e.g. reactive group X) as would be understood by one of ordinary skill in the synthesis of such compounds.

DOTP-based zwitterionic metal chelators can be synthesized using the protocol described in Scheme 1.

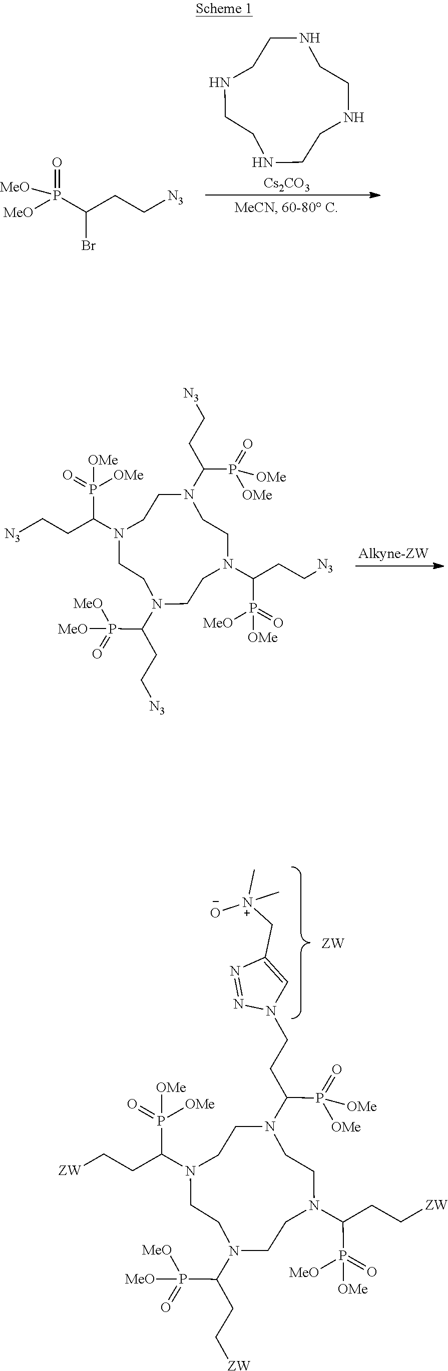

DOTP-based zwitterionic metal chelators can also be synthesized using the protocol described in Scheme 2.

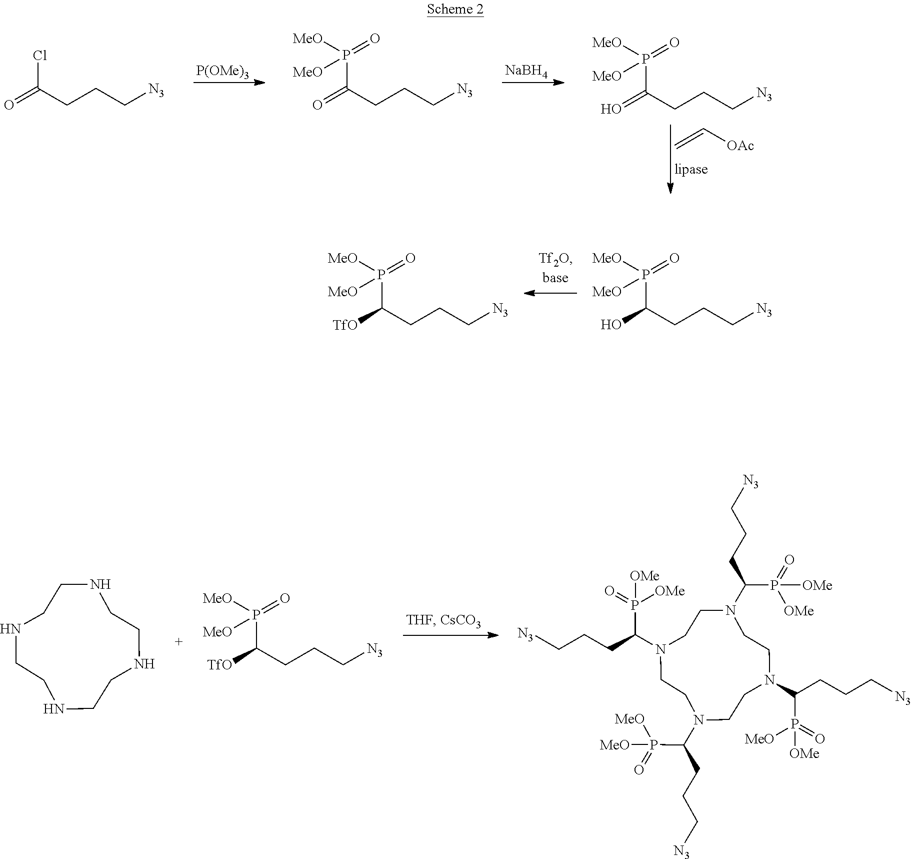

In the schemes above, the zwitterionic group can be coordinated to the metal chelator using click chemistry (i.e. through a triazole group). Nevertheless, the schemes can also be adapted to replace the azide group with a directly bound zwitterionic group.

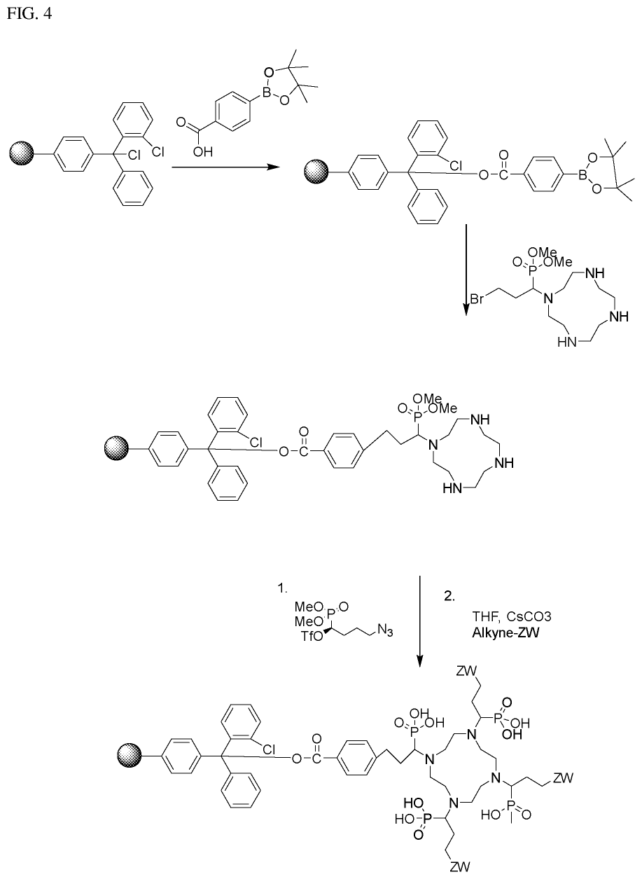

DOTP-based zwitterionic metal chelators can also be synthesized using a solid-phase protocol described in Scheme 3 (FIG. 4). Scheme 3 depicts a solid-phase synthetic scheme which produces an azide functionality to which the zwitterionic groups can be added using click-chemistry methods as described herein and as would be familiar to one of the art. Scheme 3 also permits conjugation to targeting ligands via the addition of a carboxylic acid.

In Scheme 3, the DOTP-based zwitterionic metal chelator is formed using a solid-phase synthesis. In certain embodiments the solid phase material is a resin or coated glass bead, bed, or sheet which tethers the molecule during synthesis. In certain embodiments, the solid-phase material is a CTC resin. Other resins which can be used in this synthesis include PAM resins, Wang resins, HMPB/HMBA resins, DHP resins, Wienreb aminomethyl resins, SASRIN resins, HMPB/HIMBA, Rink Amide resins, Sieber Amide resins, PAL resins, BHA resins, Merrifield resins, CHEMMATRIX resins, TENTAGEL resins, and SCAL (Safety-Catch) resins. This type of synthesis results in a compound having a free COOH that can be coupled to targeting vectors or linkers—said linkers optionally including other free COOH groups or other zwitterionic groups. This synthesis also results in azide groups which can be used to form zwitterionic groups using click-chemistry; for example, ZW groups having an N-oxide group or a sulphobetaine group bound to the DOTP ring through a triazole ring. This type of click-chemistry linking of the zwitterionic groups can also be applied to Schemes 1 and 2.

In this, N-oxide functionalities may also be synthesized by nucleophilic displacement of a suitable leaving group (such as a halogenide or a sulfonate) instead of the group R in the scheme above. The nucleophile in these conversions is an N,N-dialkylated hydroxylamine, which can be protected at the hydroxyl group.

In a particular embodiment, the DOTP-based zwitterionic metal chelators can be complexed with Bi, Pb, Zr, Cu, Ga, In, Y, Gd, Lu, Ac, Tb, Pb, Pd or other metals. In particular embodiments, the DOTP-based zwitterionic metal chelators chelate Bi3+ more tightly than commercially available metal chelators. This has the effect of minimizing the ejection of Pb-212/Bi-3+ from the chelated complex.

In still other embodiments, one or more zwitterionic groups of the DOTP-based zwitterionic metal chelator can be replaced with a targeting vector, such as cRGD, dPSMA-617, KUE, a FAP-targeting small molecule, octreotide, bombesin, or their corresponding homo- or hetero-dimers provided that DOTAM-based zwitterionic metal chelator remains zwitterionic. In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC(SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin. Alternatively, the targeting vector can be covalently attached to a reactive linking group of the chelator compound of the invention through standard coupling procedures. For example, the carboxyl or activated carboxyl group of a reactive linking group can react with a nucleophilic functionality on the targeting vector, such as an amine or alcohol derivative, to form an amide or ester linkage. Additional details for the conjugation can be found in WO 2008/017074 and in Frangioni et al. Molecular Imaging, Vol. 1(4), 354-364 (2002), each of which is incorporated herein by reference in its entirety.

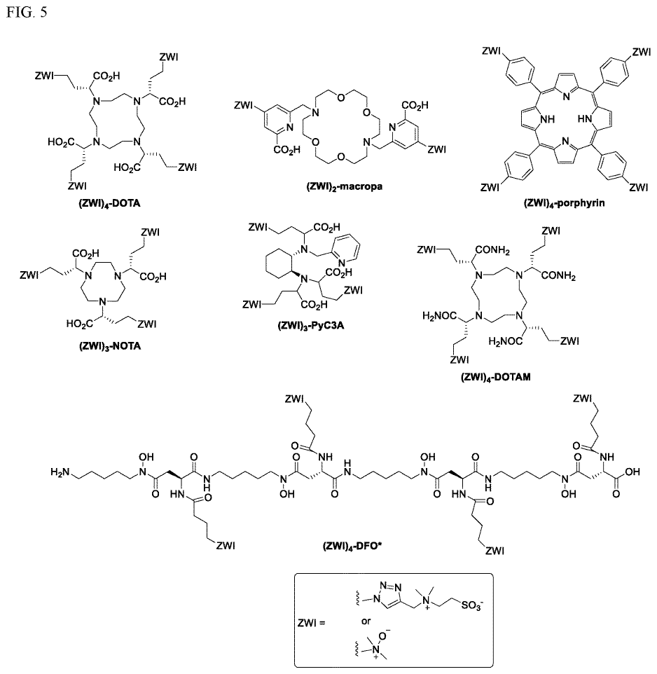

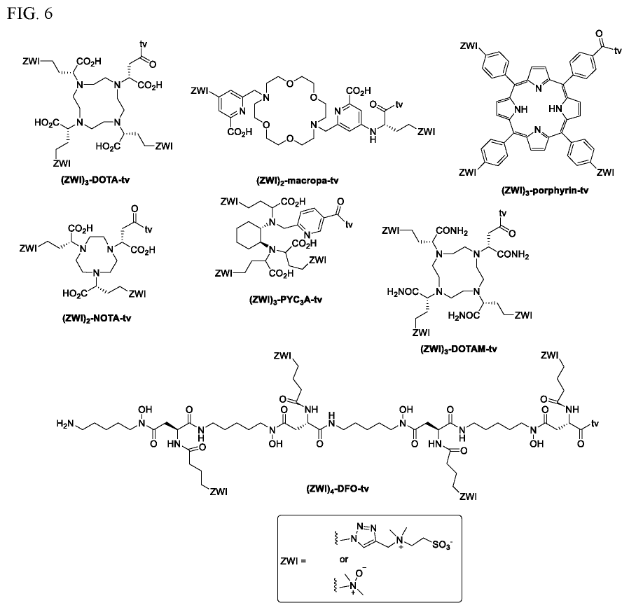

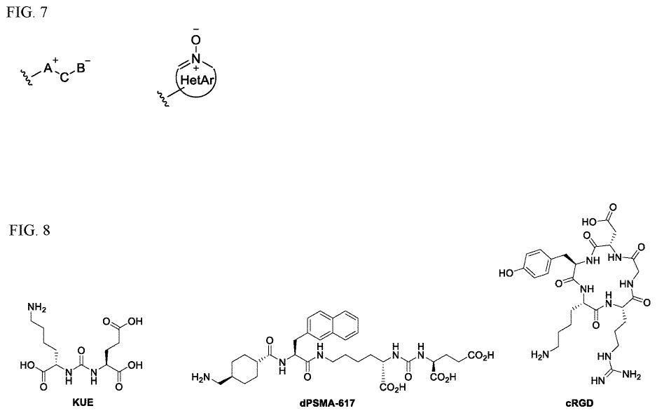

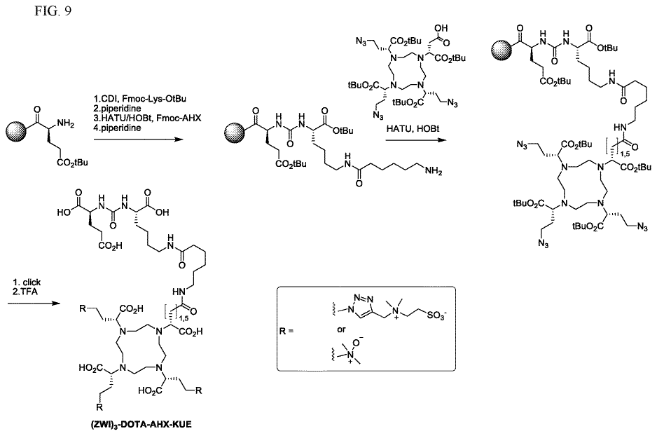

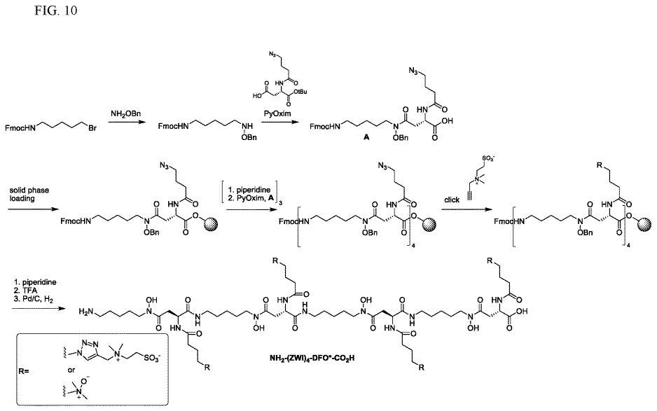

It should be apparent to those skilled in the art, that if a targeting vector replaces a zwitterionic group, that zwitterionic group can be restored by adding it to the linker between the zwitterionic metal chelator and the targeting vector. The resulting structures of the conjugates are shown in FIG. 5. The zwitterions ZWI (as defined in FIG. 2) can be separated from the chelator core structure by appropriate spacer moieties Y including alkyl, aryl or heteroaryl groups. The targeting vectors (tv) can be separated from the chelator core structure by appropriate spacer moieties W including alkyl, aryl, heteroaryl, ether, ester, amide, imine and oxime groups. The spacers Y can also contain one or more ether or amide bond or a combination of both and might contain zwitterionic groups added to the sidechains of the spacer moiety. In certain embodiments, each of W and Y may independently be absent.

In certain embodiments, the agent further comprises a PEG-moiety to alter the circulation time in blood. Such moiety can be bound to the conjugate at any suitable structural location as would be understood by one of ordinary skill in the synthesis of such compounds.

In certain embodiments, the conjugate of the DOTP-based zwitterionic metal chelator and the targeting vector has the following formula with KUE, dPSMA-617, cRGD, FAPI, octreotide, bombesin, or as the targeting vector (tv):

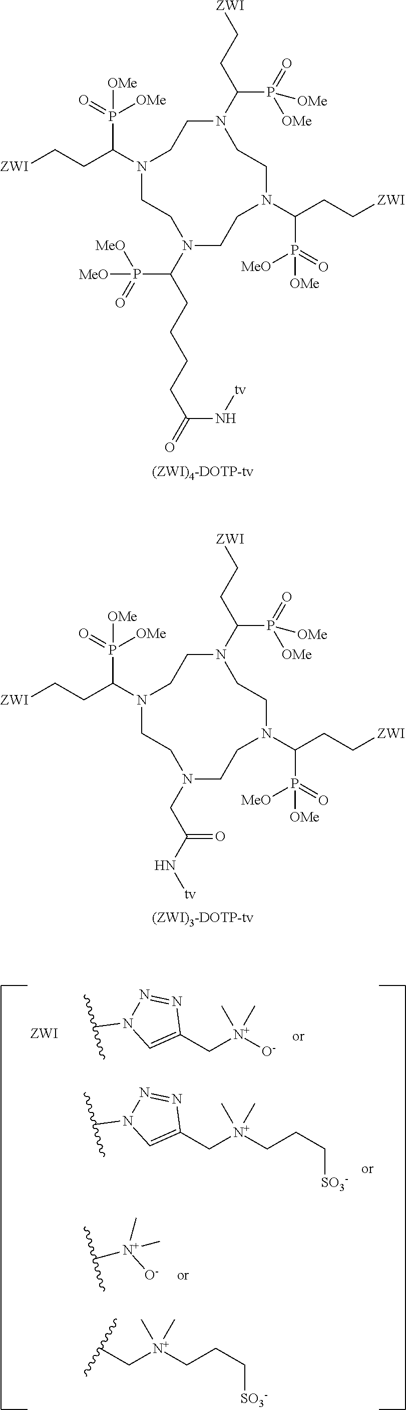

In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC(SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin.

In some cases, one of ordinary skill in the art would understand that one or more zwitterionic groups can be replaced with another moiety which could also impart hydrophilicity to the chelator. Such hydrophilicity-imparting groups may be substituted, added, or exchanged for alternative moieties that maintain or increase aqueous solubility and hydrophilicity of the molecule (e.g., a chelator or conjugate). Non-limiting examples of suitable hydrophilic groups include poly(ethylene glycol) (PEG) chains; saccharides and sugar derivatives (e.g., glucose, galactose, mannose, sialic acid); polyols and hydroxylated moieties (e.g., glycerol, sorbitol, mannitol, xylitol, diols, triols); carboxylic acids and carboxylates; sulfonic acids and sulfonates; sulfate groups; phosphate and phosphonate groups; amide, urea, and carbamate functionalities; amine oxides; sulfoxides; betaines (e.g., carboxybetaine, sulfobetaine); quaternary ammonium groups; guanidinium-containing groups; and polar heterocycles (e.g., morpholine, piperazine, N-vinylpyrrolidone). Such moieties may be introduced in protected or salt forms and can be tethered via suitable linkers to achieve the desired hydrophilicity, charge, and solubility characteristics.

### MacroPaP/MacroPaN—Based Zwitterionic Metal Chelators

In one aspect, the invention provides a zwitterionic metal chelator based on macropa in which —COOH groups are replaced with —PO(OH)₂ groups or —NR′₂ groups. Such zwitterionic metal chelators, which may be referred to as macropaP or macropaN, respectively, and include the following:

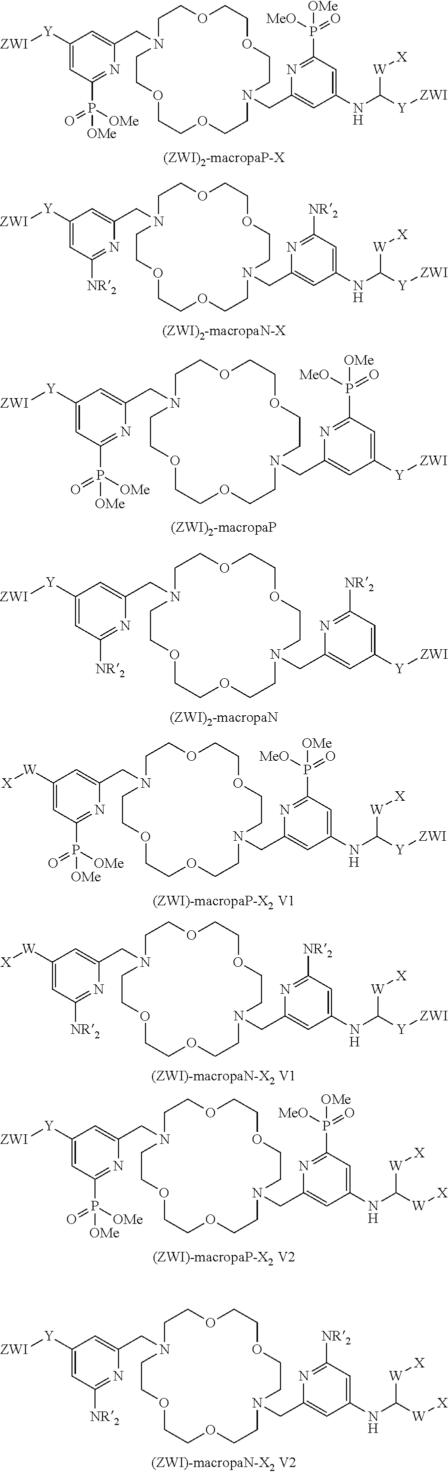

in which ZWI represents a zwitterionic group; each instance of W and Y independently represent a linking group, and each instance of X represents a reactive group and/or a targeting vector. In some embodiments, the zwitterionic group (ZWI) is an ammonium sulphobetaine group or an N-oxide group. In some embodiments, the zwitterionic group is optionally coordinated through click-chemistry (a triazole group). In the case of zwitterionic macropaN chelators, each instance of R′ independently represents a hydrogen or alkyl group.

In such embodiments, a macropaP/macropaN-based zwitterionic metal chelator includes 1 or 2 zwitterionic groups as defined in FIG. 3 and 1 or 2 reactive groups X for conjugation to targeting vectors or other molecules or materials.

In certain embodiments, ZWI represents an ammonium sulfobetaine group. In other embodiments ZWI represents an N-oxide group. This reactive group can be a carboxylic acid, an activated derivative of a carboxylic acid, such as an NHS ester, a sulfo-NHS ester, a PFP ester, a HOBt ester, a HOAt ester, a TFP ester, an acid anhydride, an acid azide or an acid halide. The reactive group X can also be an amine, azide, alkyne, alkene, ketone, aldehyde, alcohol, phenol, maleimide, thiol, phosphane or a tetrazine. The zwitterionic groups ZWI and the reactive groups X can be separated from the chelator core structure by appropriate spacer moieties W and Y including alkyl, aryl or heteroaryl groups. In certain embodiments, each of W and Y may independently be absent.

Representative ZWI groups include those shown in FIG. 2 and, for example, those shown below:

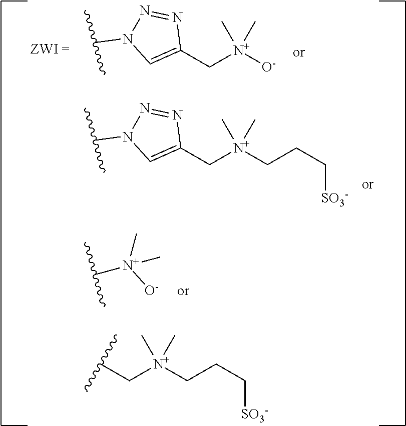

In certain embodiments, a macropaP/macropaN-based zwitterionic metal chelator contains stereogenic centers, any of these might have R or S-configuration. The macropaP/macropaN-based zwitterionic metal chelator might be a single stereoisomer or might be a mixture of stereoisomers. The macropaP/macropaN-based zwitterionic metal chelator includes 1, 2, 3, or 4 zwitterionic groups as defined in FIG. 3 and 1, 2 or 3 reactive groups X for conjugation to targeting vectors or other molecules or materials. This reactive group can be a carboxylic acid, an activated derivative of a carboxylic acid, such as an NHS ester, a sulfo-NHS ester, a PFP ester, a HOBt ester, a HOAt ester, a TFP ester, an acid anhydride, an acid azide or an acid halide. The reactive group X can also be an amine, azide, alkyne, alkene, ketone, aldehyde, alcohol, phenol, maleimide, thiol, phosphane or a tetrazine. The zwitterionic groups ZWI and the reactive groups X can be separated from the chelator core structure by appropriate spacer moieties W and Y including alkyl, aryl or heteroaryl groups. In particular embodiments, the macropaP/macropaN-based zwitterionic metal chelator includes 1, 2, 3, or 4 zwitterionic groups and 4 carboxylate groups for complexation to the metal or metal isotope. In still other embodiments, the macropaP/macropaN-based zwitterionic metal chelator includes 1, 2, 3, or 4 zwitterionic groups, 4 carboxylate groups for complexation to the metal or metal isotope, and one or more targeting vectors. These targeting vectors can be bound to the chelator at any suitable structural location (e.g. reactive group X) as would be understood by one of ordinary skill in the synthesis of such compounds.

In particular embodiments, the macropaP-based zwitterionic metal chelator has the formula:

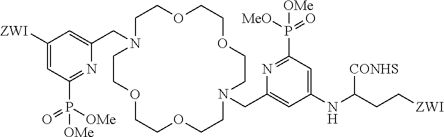

wherein ZWI represents a zwitterionic group. In certain embodiments, ZWI represents an ammonium sulfobetaine group or an N-Oxide group, optionally coordinated through click-chemistry (a triazole group). In particular embodiments, the macropaN-based zwitterionic metal chelator has the formula:

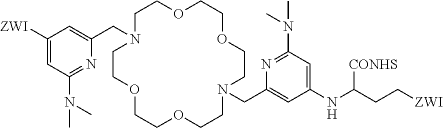

wherein ZWI represents a zwitterionic group. In certain embodiments, ZWI represents an ammonium sulfobetaine group or an N-Oxide group, optionally coordinated through click-chemistry (a triazole group).

MacropaP/macropaN-based zwitterionic metal chelators can be synthesized using the protocol described in Scheme 4.

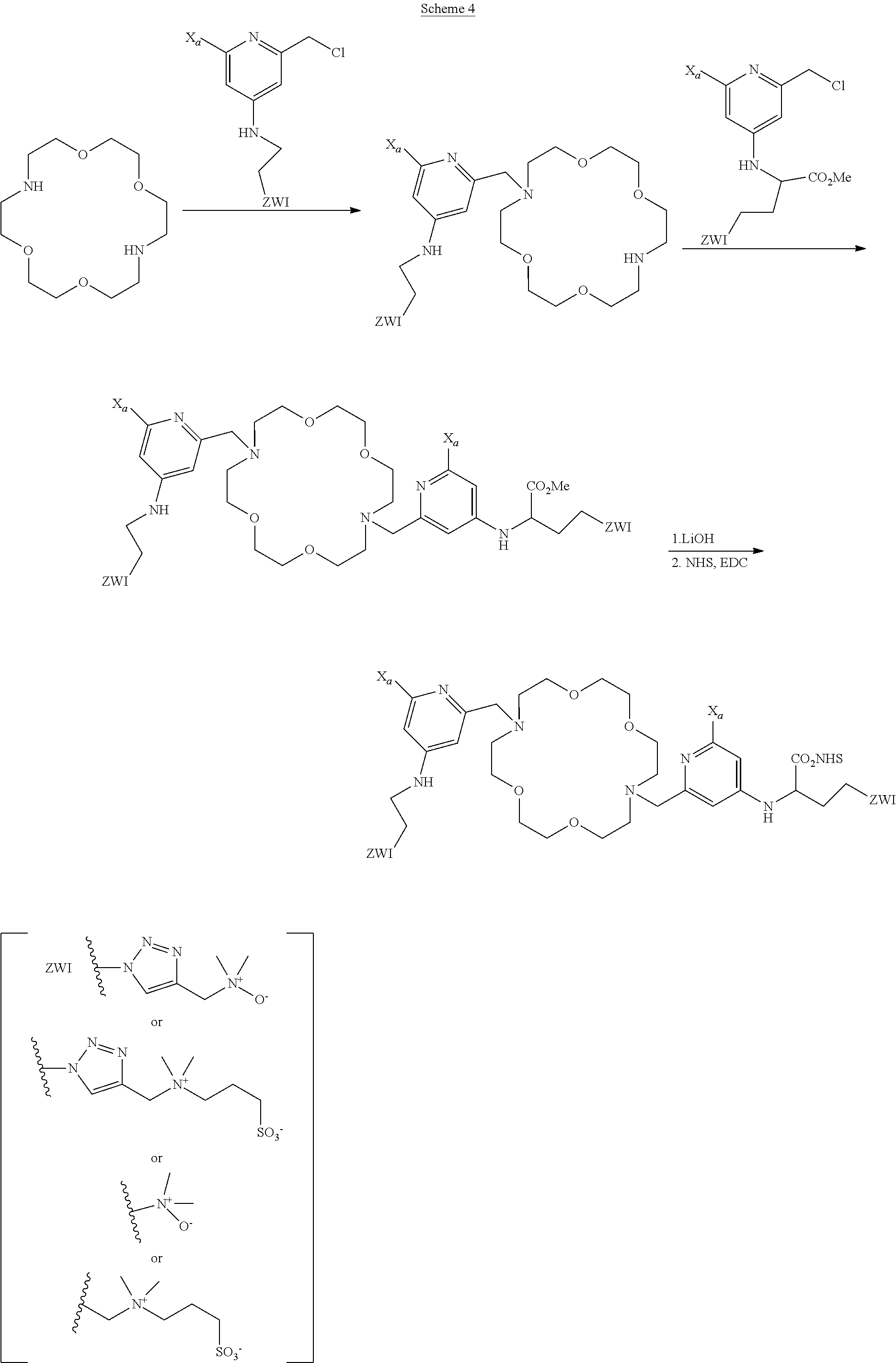

wherein Xₐ represents a protected phosphate or amine/amide group (i.e. —PO(OMe)₂-NMe₂; and ZWI represents a zwitterionic group. wherein X_{b} represents a carboxyl group or a phosphate, optionally protected with alkoy groups in place of the hydroxy groups; and ZWI represents a zwitterionic group. Although scheme 4 shows the production (ZW)₂-MacropaP/MacropaN-zwitterionic metal chelator with direct of ZWI groups, the use of click chemistry schemes (through a triazole group) can also be achieved using similar synthetic schemes. Similarly, MacropaP/MacropaN—based zwitterionic metal chelators with fewer different numbers of zwitterionic groups can also be achieved using similar synthetic schemes.

In a particular embodiment, the MacropaP/macropaN-based zwitterionic metal chelators can be complexed with Ac³⁺, Bi³⁺, or similar metal cations.

In still other embodiments, one or more zwitterionic groups of the MacropaP/macropaN-based zwitterionic metal chelator can be replaced with a targeting vector, such as cRGD, dPSMA-617, KUE, a FAP-targeting small molecule, octreotide, bombesin, or their corresponding homo- or heterodimers, provided that MacropaP/macropaN-based zwitterionic metal chelator remains zwitterionic. In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC(SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin. Alternatively, the targeting vector can be covalently attached to a reactive linking group of the chelator compound of the invention through standard coupling procedures. For example, the carboxyl or activated carboxyl group of a reactive linking group can react with a nucleophilic functionality on the targeting vector, such as an amine or alkohol derivative, to form an amide or ester linkage. Additional details for the conjugation can be found in WO 2008/017074 and in Frangioni et al. Molecular Imaging, Vol. 1(4), 354-364 (2002), each of which is incorporated herein by reference in its entirety.

It should be apparent to those skilled in the art, that if a targeting vector replaces a zwitterionic group, that zwitterionic group can be restored by adding it to the linker between the zwitterionic metal chelator and the targeting vector. The zwitterions ZWI can be separated from the chelator core structure by appropriate spacer moieties Y including alkyl, aryl or heteroaryl groups. The targeting vectors (tv) can be separated from the chelator core structure by appropriate spacer moieties W including alkyl, aryl, heteroaryl, ether, ester, amide, imine and oxime groups. The spacers Y can also contain one or more ether or amide bond or a combination of both and might contain zwitterionic groups added to the sidechains of the spacer moiety. In certain embodiments, each of W and Y may independently be absent.

MacropaP/macropaN-based zwitterionic metal chelators can also be synthesized using a modification of the solid-phase protocol described in Scheme 3 (FIG. 4).

Scheme 3 depicts a solid-phase synthetic scheme which produces an azide functionality to which the zwitterionic groups can be added using click-chemistry methods as described herein and as would be familiar to one of the art. Scheme 3 also permits conjugation to targeting ligands via the addition of a carboxylic acid.

In either synthesis, N-oxide functionalities may also be synthesized by nucleophilic displacement of a suitable leaving group (such as a halogenide or a sulfonate) instead of the group R in the scheme above. The nucleophile in these conversions is an N,N-dialkylated hydroxylamine, which can be protected at the hydroxyl group.

Conjugates of macropaP/macropaN-based zwitterionic chelators and targeting vectors (tv) include, but are not limited to:

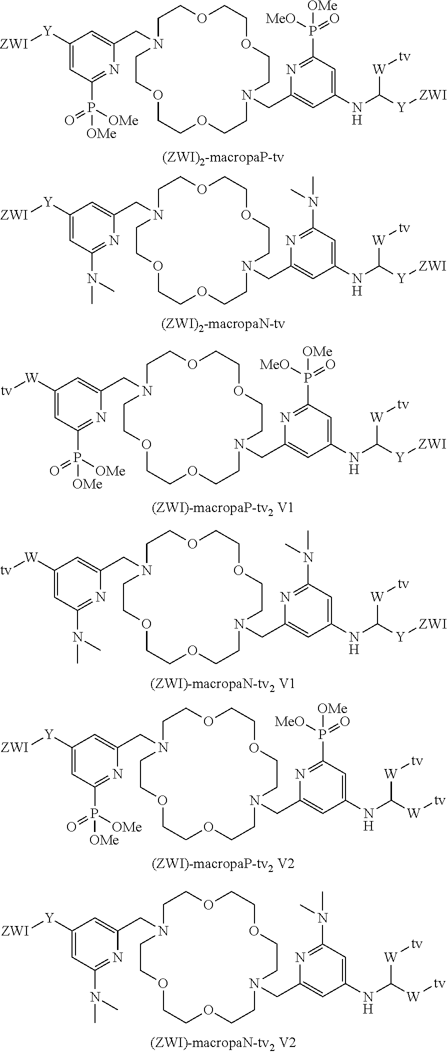

In certain embodiments, the agent further comprises a PEG-moiety to alter the circulation time in blood. Such moiety can be bound to the conjugate at any suitable structural location as would be understood by one of ordinary skill in the synthesis of such compounds.

In certain embodiments, the conjugate of the MacropaP/macropaN-based zwitterionic metal chelator and the targeting vector has the following formula with KUE, dPSMA-617, cRGD, FAPI, octreotide, bombesin, or as the targeting vector (tv).

In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC(SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin.

In some cases, one of ordinary skill in the art would understand that one or more zwitterionic groups can be replaced with another moiety which could also impart hydrophilicity to the chelator. Such hydrophilicity-imparting groups may be substituted, added, or exchanged for alternative moieties that maintain or increase aqueous solubility and hydrophilicity of the molecule (e.g., a chelator or conjugate). Non-limiting examples of suitable hydrophilic groups include poly(ethylene glycol) (PEG) chains; saccharides and sugar derivatives (e.g., glucose, galactose, mannose, sialic acid); polyols and hydroxylated moieties (e.g., glycerol, sorbitol, mannitol, xylitol, diols, triols); carboxylic acids and carboxylates; sulfonic acids and sulfonates; sulfate groups; phosphate and phosphonate groups; amide, urea, and carbamate functionalities; amine oxides; sulfoxides; betaines (e.g., carboxybetaine, sulfobetaine); quaternary ammonium groups; guanidinium-containing groups; and polar heterocycles (e.g., morpholine, piperazine, N-vinylpyrrolidone). Such moieties may be introduced in protected or salt forms and can be tethered via suitable linkers to achieve the desired hydrophilicity, charge, and solubility characteristics.

### PYT—Based Zwitterionic Metal Chelators

In one aspect, the invention provides a zwitterionic metal chelator based on 3,6,10,13-tetraaza-1,8(2,6)-dipyridinacyclotetradecaphane-3,6,10,13-tetraacetic acid (PYTA). Such zwitterionic metal chelators, which may be referred to as PYT-Based chelators, include the following:

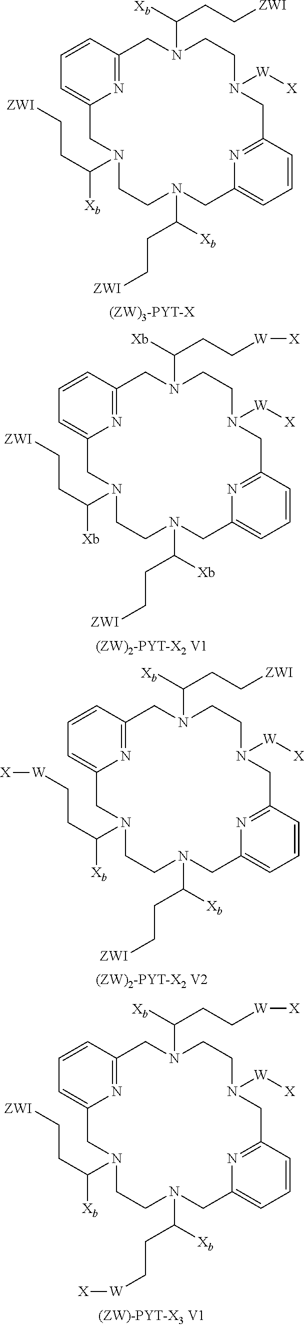

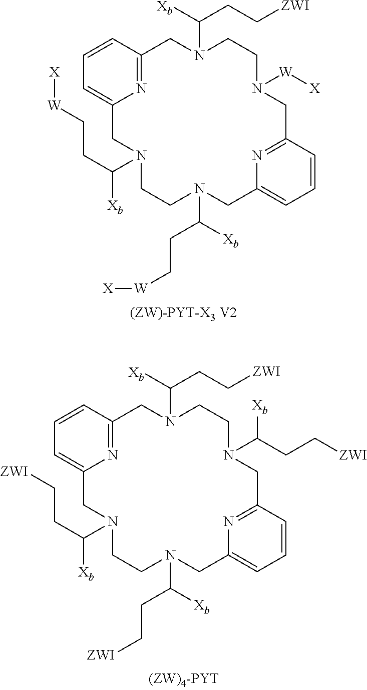

in which ZWI represents a zwitterionic group; each instance of W and Y independently represent a linking group, each instance of X_{b} may be —COOH, —PO(OH)₂, or —NR′₂; each instance of R′ is independently hydrogen or an alkyl group; each instance of X independently represents a reactive group and/or a targeting vector. In certain embodiments, ZWI represents an ammonium sulfobetaine group. In other embodiments ZWI represents an N-oxide group. In embodiments in which each X_{b} is —COOH, the chelator may be referred to as a PYTA-based chelator. In embodiments in which each X_{b} is —PO(OH)₂, the chelator may be referred to as a PYTP-based chelator.

In such embodiments, a PYT-based zwitterionic metal chelator includes 1, 2, or 3 zwitterionic groups 1-3 reactive groups X for conjugation to targeting vectors or other molecules or materials.

In certain embodiments, ZWI represents an ammonium sulfobetaine group. In other embodiments ZWI represents an N-oxide group. This reactive group can be a carboxylic acid, an activated derivative of a carboxylic acid, such as an NHS ester, a sulfo-NHS ester, a PFP ester, a HOBt ester, a HOAt ester, a TFP ester, an acid anhydride, an acid azide or an acid halide. The reactive group X can also be an amine, azide, alkyne, alkene, ketone, aldehyde, alcohol, phenol, maleimide, thiol, phosphane or a tetrazine. The zwitterionic groups ZWI and the reactive groups X can be separated from the chelator core structure by appropriate spacer moieties W and Y including alkyl, aryl or heteroaryl groups. In certain embodiments, each of W and Y may independently be absent.

Representative ZWI groups include those shown in FIG. 2 and, for example, those shown below:

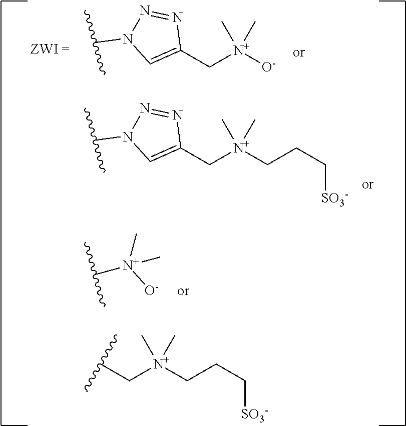

In certain embodiments, a PYT-based zwitterionic metal chelator contains stereogenic centers, any of these might have R or S-configuration. The PYT-based zwitterionic metal chelator might be a single stereoisomer or might be a mixture of stereoisomers. The PYT-based zwitterionic metal chelator includes 1, 2, 3, or 4 zwitterionic groups as defined in FIG. 3 and 1, 2 or 3 reactive groups X for conjugation to targeting vectors or other molecules or materials. This reactive group can be a carboxylic acid, an activated derivative of a carboxylic acid, such as an NHS ester, a sulfo-NHS ester, a PFP ester, a HOBt ester, a HOAt ester, a TFP ester, an acid anhydride, an acid azide or an acid halide. The reactive group X can also be an amine, azide, alkyne, alkene, ketone, aldehyde, alcohol, phenol, maleimide, thiol, phosphane or a tetrazine. The zwitterionic groups ZWI and the reactive groups X can be separated from the chelator core structure by appropriate spacer moieties W and Y including alkyl, aryl or heteroaryl groups. In particular embodiments, the PYT-based zwitterionic metal chelator includes 1, 2, 3, or 4 zwitterionic groups and 4 carboxylate groups for complexation to the metal or metal isotope. In still other embodiments, the PYT-based zwitterionic metal chelator includes 1, 2, 3, or 4 zwitterionic groups, 4 carboxylate groups for complexation to the metal or metal isotope, and one or more targeting vectors. These targeting vectors can be bound to the chelator at any suitable structural location (e.g. reactive group X) as would be understood by one of ordinary skill in the synthesis of such compounds.

In particular embodiments, the PYT-based zwitterionic metal chelator has the formula:

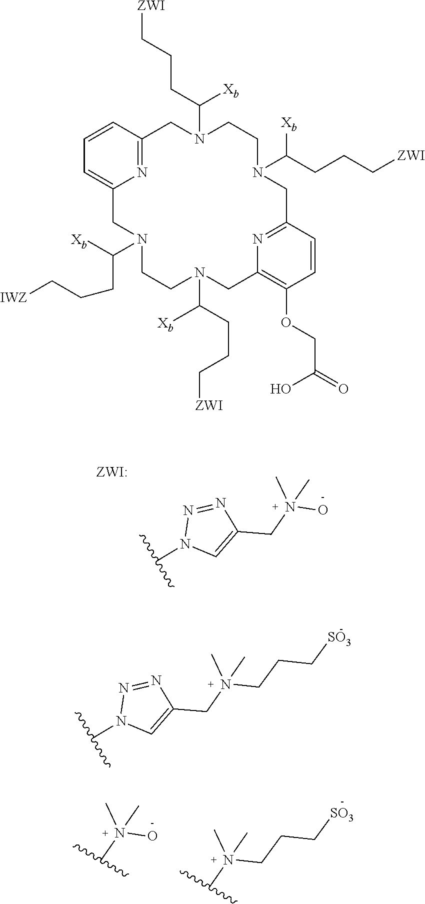

wherein ZWI represents a zwitterionic group and X_{b} may be —COOH, —PO(OH)₂, or —NR′₂; wherein each instance of R′ is independently hydrogen or an alkyl group. In certain embodiments, ZWI represents an ammonium sulfobetaine group.

PYT-based zwitterionic metal chelators can be synthesized using the protocol described in Scheme 5.

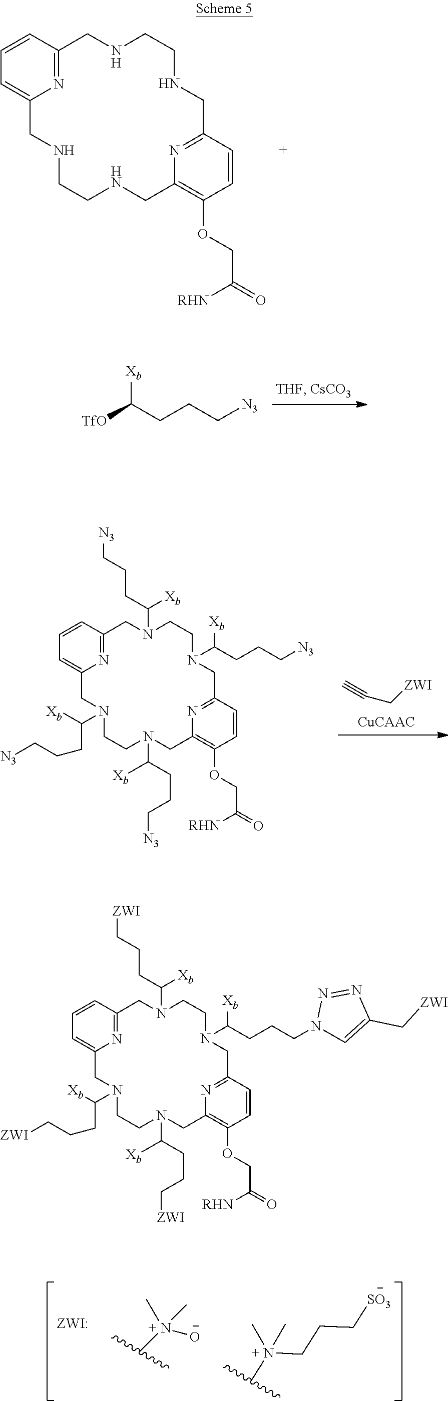

wherein X_{b} represents a carboxyl group or a phosphate, optionally protected with alkoy groups in place of the hydroxy groups; and ZWI represents a zwitterionic group. Although scheme 5 shows the production (ZW)₄-PYT-zwitterionic metal chelator using a click-chemistry scheme, attachment of ZWI groups without the triazole group can also be achieved using similar synthetic schemes. Similarly, PYT-based zwitterionic metal chelators with fewer than 4 zwitterionic groups can also be achieved using similar synthetic schemes.

PYT-based zwitterionic metal chelators can also be synthesized using a solid-phase modification of the protocol described in Scheme 5, In particular, the chelator in the first step can be modified by coupling the chelator to a solid phase material (such as a CTC resin) through the —CONHR group. Similar modifications can be used to couple the chelator to a targeting ligand in the same position through standard amide coupling reactions. PYT-based zwitterionic metal chelators can also be synthesized using a modification of the solid-phase protocol described in Scheme 3 (FIG. 4).

In a particular embodiment, the PYT-based zwitterionic metal chelators can be complexed with Ac³⁺, Bi³⁺, or similar metal cations.

In still other embodiments, one or more zwitterionic groups of the PYT-based zwitterionic metal chelator can be replaced with a targeting vector, such as cRGD, dPSMA-617, KUE, a FAP-targeting small molecule, octreotide, bombesin, or their corresponding homo- or heterodimers, provided that PYT-based zwitterionic metal chelator remains zwitterionic. In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC(SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin. Alternatively, the targeting vector can be covalently attached to a reactive linking group of the chelator compound of the invention through standard coupling procedures. For example, the carboxyl or activated carboxyl group of a reactive linking group can react with a nucleophilic functionality on the targeting vector, such as an amine or alkohol derivative, to form an amide or ester linkage. Additional details for the conjugation can be found in WO 2008/017074 and in Frangioni et al. Molecular Imaging, Vol. 1(4), 354-364 (2002), each of which is incorporated herein by reference in its entirety.

It should be apparent to those skilled in the art, that if a targeting vector replaces a zwitterionic group, that zwitterionic group can be restored by adding it to the linker between the zwitterionic metal chelator and the targeting vector. The zwitterions ZWI can be separated from the chelator core structure by appropriate spacer moieties Y including alkyl, aryl or heteroaryl groups. The targeting vectors (tv) can be separated from the chelator core structure by appropriate spacer moieties W including alkyl, aryl, heteroaryl, ether, ester, amide, imine and oxime groups. The spacers Y can also contain one or more ether or amide bond or a combination of both and might contain zwitterionic groups added to the sidechains of the spacer moiety. In certain embodiments, each of W and Y may independently be absent.

In either synthesis, N-oxide functionalities may also be synthesized by nucleophilic displacement of a suitable leaving group (such as a halogenide or a sulfonate) instead of the group R in the scheme above. The nucleophile in these conversions is an N,N-dialkylated hydroxylamine, which can be protected at the hydroxyl group.

Conjugates of PYT-based zwitterionic chelators and targeting vectors (tv) include, but are not limited to:

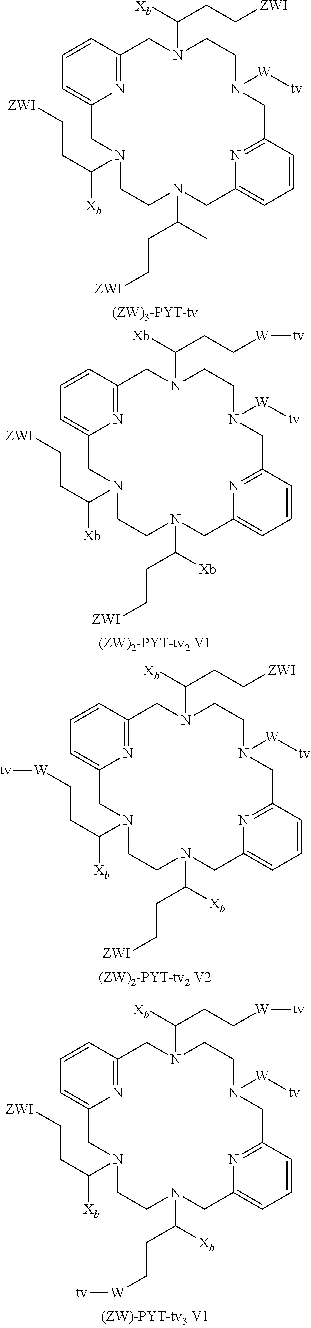

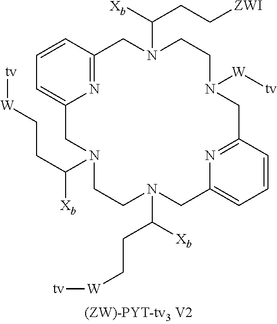

In certain embodiments, the agent further comprises a PEG-moiety to alter the circulation time in blood. Such moiety can be bound to the conjugate at any suitable structural location as would be understood by one of ordinary skill in the synthesis of such compounds.

In such embodiments, the conjugate of the PYT-based zwitterionic metal chelator and the targeting vector has KUE, dPSMA-617, cRGD, FAPI, octreotide, bombesin, or as the targeting vector (tv).

In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC(SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin.

In some cases, one of ordinary skill in the art would understand that one or more zwitterionic groups can be replaced with another moiety which could also impart hydrophilicity to the chelator. Such hydrophilicity-imparting groups may be substituted, added, or exchanged for alternative moieties that maintain or increase aqueous solubility and hydrophilicity of the molecule (e.g., a chelator or conjugate). Non-limiting examples of suitable hydrophilic groups include poly(ethylene glycol) (PEG) chains; saccharides and sugar derivatives (e.g., glucose, galactose, mannose, sialic acid); polyols and hydroxylated moieties (e.g., glycerol, sorbitol, mannitol, xylitol, diols, triols); carboxylic acids and carboxylates; sulfonic acids and sulfonates; sulfate groups; phosphate and phosphonate groups; amide, urea, and carbamate functionalities; amine oxides; sulfoxides; betaines (e.g., carboxybetaine, sulfobetaine); quaternary ammonium groups; guanidinium-containing groups; and polar heterocycles (e.g., morpholine, piperazine, N-vinylpyrrolidone). Such moieties may be introduced in protected or salt forms and can be tethered via suitable linkers to achieve the desired hydrophilicity, charge, and solubility characteristics.

### Targeting Vectors

In some embodiments of the metal chelators of the invention, one or more zwitterionic groups of the zwitterionic metal chelator can be replaced with a targeting vector, such as cRGD, a PSMA binding vector, such as PSMA-617 or KUE, a GPI-derivative, a FAP-targeting molecule (FAP-inhibitor or FAPI), octreotide, a bombesin analog, or their corresponding homo- and hetero-dimers provided that zwitterionic metal chelator remains zwitterionic.

In certain embodiments, the targeting vectors are agonists or antagonists of the target, including, but not limited to, agonists or antagonists of integrins, agonists or antagonists of FAP, agonists or antagonists of PSMA, agonists or antagonists of somatostatin receptor, and agonists or antagonists of GRPR. In still other embodiments, the targeting vector may be a derivative of the targeting vector, including, but not limited to, derivatives of cRGD, derivatives of FAP, derivatives of PSMA-binding vectors, derivatives of octreotide, and derivatives of bombesin.

In certain embodiments, the targeting vector can be attached to the metal chelators of the invention through an ester linkage. In particular embodiments, the targeting vector can be attached to the metal chelators by reaction with a carboxyl-substituted phenyl group. In still other embodiments, the targeting vector can be attached to the metal chelators by the inclusion of one or more spacer groups or linker groups, wherein the spacer or linker groups may optionally include additional free carboxyl groups or zwitterionic groups for further functionality. In particular embodiments, the targeting vector is attached to the metal chelators by one or more linker groups and through an ester linkage to a carboxyl-substituted phenyl group.

In certain embodiments of the invention, the targeting ligand according to the invention can be a cyclic-RGD having the following structure:

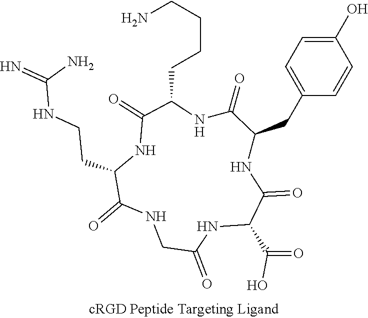

In certain embodiments, the FAPI-targeting ligand includes NH2-FAPI-74. Specific details regarding NH2-FAPI-74 are described in: Linder et al., “Radioligands Targeting Fibroblast Activation Protein (FAP),” the entirety of which is incorporated herein by reference. In general, NH2-FAPI-74 has the following structure: the FAPI-targeting ligand includes NH₂-FAPI-74

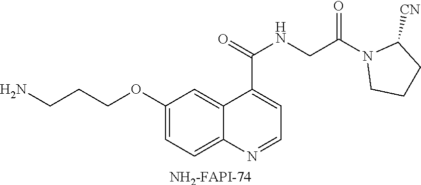

In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC(SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin.

In certain embodiments, the targeting ligand includes one or more derivatives of bombesin, including, but not limited to, BIM26226, JMV594, JMV641, JMV736, JMV717, JMV845, and JMV542. Additional bombesin derivatives, including bombesin agonists, bombesin antagonists, and bombesin pseudopeptides can be found in Azay et al., Peptides, Vol 19, No. 1, pp 57-63, 1988—which is incorporated herein by reference.

In a particular aspect, the invention provides a zwitterionic metal chelator conjugated to a targeting vector for prostate specific membrane antigen (PSMA). For example, a targeting vector based on (((S)-5-((S)-2-((1r,4S)-4-(aminomethyl)cyclohexane-1-carboxamido)-3-(naphthalen-2-yl)propanamido)-1-carboxypentyl)carbamoyl)-L-glutamic acid, which is a derivative of PSMA-617 or Vipivotide tetraxetan. This PSMA-targeting vector is called dPSMA-617 (for “derivative of PSMA-617) in this disclosure.

An alternative PSMA-targeting vector is (((S)-5-amino-1-carboxypentyl)carbamoyl)-L-glutamic acid (KUE).

The structures of dPSMA-617 and KUE are shown in FIG. 3.

In such embodiments, a KUE or dPSMA-617-conjugated zwitterionic metal chelator includes 1-4 zwitterionic groups. In particular embodiments, the KUE or dPSMA-617-conjugated zwitterionic metal chelator includes 1-3 zwitterionic groups.

In particular embodiment, the KUE or PSMA-617-conjugated zwitterionic metal chelators can be complexed with Ga³⁺, Cu²⁺, Lu³⁺, Zr⁴⁺, Mn²⁺, Mn³⁺, Pb²⁺, Tb³⁺, Gd³⁺, or similar metal cations.

In certain embodiments, the targeting ligand includes a phosphinate mimic of N-acetylaspartylglutamic acid (GPI) or a derivative thereof. Particular derivatives of GPI can be found in U.S. Provisional Ser. No. 63/858,228 filed Aug. 5, 2025.

In certain embodiments, the GPI targeting ligand may serve as the pharmacophore component that provides selective binding to prostate-specific membrane antigen (PSMA). The GPI targeting ligand may comprise a glutamate portion linked to a phosphoramidate portion, forming a bifunctional binding unit that engages multiple sites within the PSMA active site.

In certain embodiments, the GPI targeting ligand comprises a general chemical structure, where n=0 to 3 for Regions A and D:

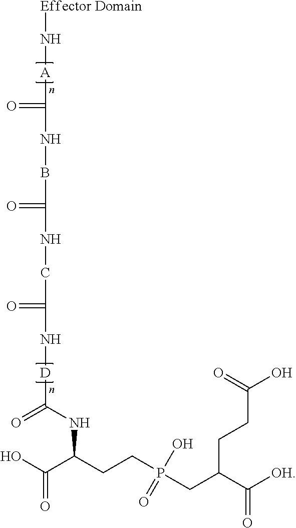

Three key examples of GPI targeting ligand includes:

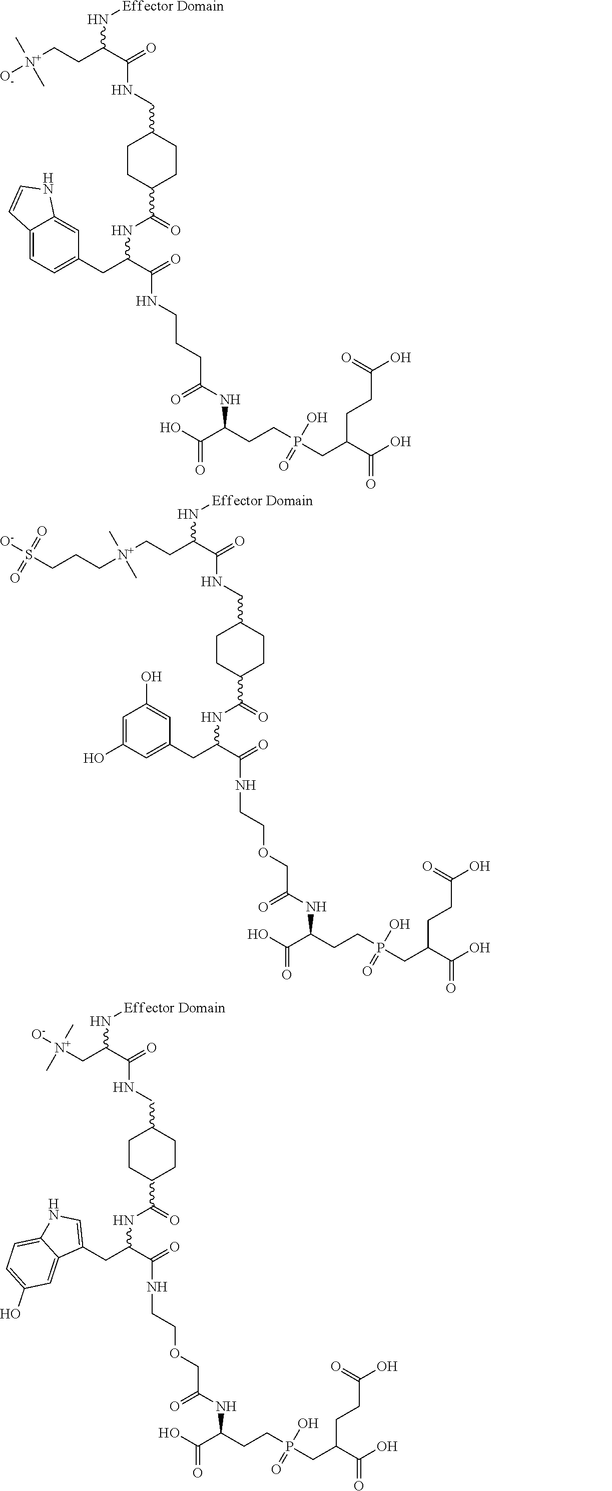

In certain embodiments, the targeting ligand includes a dimer or a multimer of one or more of KUE, dPSMA-617, GPI-derivatives, or a derivative thereof.

In general, the structure of octreotide (SEQ ID No: 5) is:

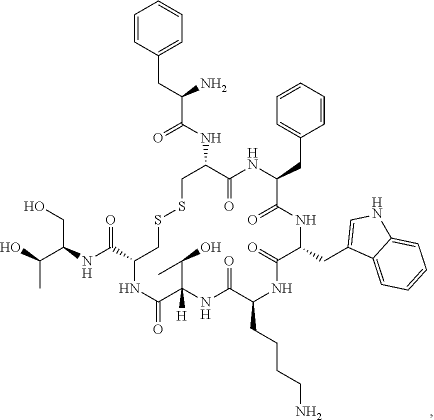 [SMILES: C[C@@H](O)[C@@H]1NC(=O)[C@H](CCCCN)NC(=O)[C@@H](Cc2c[nH]c3ccccc23)NC(=O)[C@H](Cc2ccccc2)NC(=O)[C@@H](NC(=O)[C@H](N)Cc2ccccc2)CSSC[C@@H](C(=O)N[C@H](CO)[C@@H](C)O)NC1=O]

or a derivative thereof.

In general, the structure of a bombesin analog (SEQ ID No: 6) is:

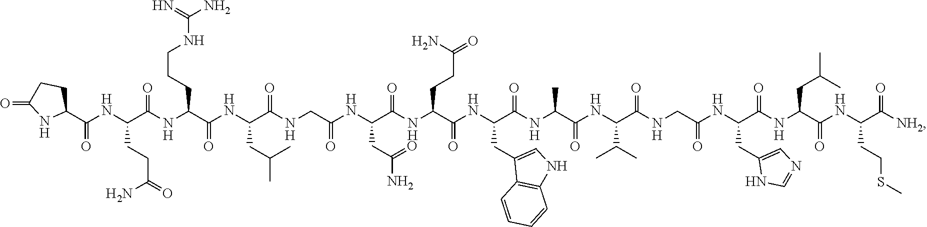 [SMILES: CSCC[C@H](NC(=O)[C@H](CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@H](CC(N)=O)NC(=O)CNC(=O)[C@H](CC(C)C)NC(=O)[C@H](CCCNC(=N)N)NC(=O)[C@H](CCC(N)=O)NC(=O)[C@@H]1CCC(=O)N1)C(C)C)C(N)=O]

or a derivative thereof.

The synthetic protocol described in Schemes 1 and 2 can be used to produce conjugated zwitterionic metal chelators based on PCTA or amide-PCTA using cRGD, a FAP-targeting molecule, octreotide, or dimers including a combination of cRGD, dPSMA-617, a FAP-targeting molecule, octreotide, and a bombesin analog. In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC(SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB 1) for diagnosing/treating tumors of epithelial origin. Depending on the reactive group of the zwitterionic metal chelator other common chemical methods may be used to conjugate the targeting vector, such as azide-alkyne-cycloadditions, nucleophilic displacements, Diels-Alder-reactions, urea and urethane formations, thiol-ene conjugations or similar conversions. For some of these conversions, derivatives of KUE,dPSMA-617, or GPI with other reactive functional groups can be used. For example, a conjugation via a copper-catalyzed azide-alkyne-cycloaddition could require either an alkyne or an azide group as the reactive group at the zwitterionic chelator and a matching alkyne or azide group as the reactive group at the targeting vector.

In certain embodiments, the targeting ligand can further include a molecular scaffold moiety to which the binding moiety and other groups can attach. In certain embodiments, the molecular scaffold moiety includes a carboxyl-substituted phenyl group which can form an ester linkage to a targeting ligand—either directly or through additional linker groups as discussed herein. In other embodiments, the molecule scaffold can bear one or more of the following: (1) a moiety designed to react with the reactive linking group of the dye to form a covalent bond, (2) a charge balancing moiety, such as any of the ionic groups described herein, and (3) a moiety that binds to the biological target. An example of a molecular scaffold is an adamantane derivative, such as described in U.S. Pat. App. Pub. No. 2006/0063834, which is incorporated herein by reference in its entirety, and illustrates the preparation of a targeting ligand that incorporates an adamantane scaffold. Specifically, the adamantane core holds (1) an amino group capable of reacting with the dye compounds, (2) a charge-balancing moiety that will neutralize a negative charge on the dye molecule, and (3) two moieties that bind to the biological target PSMA. For a description of moieties that bind to PSMA, see, Humblet, V. et al. Mol. Imaging, 2005, 4: 448-62; Misra P. et al. J. Nucl. Med. 2007, 48: 1379-89; Chen, Y., et al. J. Med. Chem, 2008, 51: 7933-43; Chandran, S. S., et al. Cancer Biol. Ther., 2008, 7:974-82; Banerjee, S. R., J. Med. Chem. 2008, 51: 4504-17; Mease, R. C., et al. Clin. Cancer Res., 2008, 14:3036-43; Foss, C. A. et al. Clin. Cancer. Res., 2005, 11:4022-8, each of which is incorporated herein by reference in its entirety. A flexible linker may function as a flexible spacer component that positions the zwitterionic chelator and any associated targeting moieties by setting the approach vector and separation distance needed for pocket engagement between the targeting moiety and the zwitterionic chelator. The flexible nature of the flexible linker may minimize steric strain while maximizing binding interactions with the target protein and maintaining optimal metal coordination geometry of the zwitterionic chelator.

The flexible linker may comprise polyethylene glycol units that provide flexibility and hydrophilicity to the overall zwitterionic chelator-based molecular construct. Polyethylene glycol units may offer biocompatibility and may reduce immunogenicity of the zwitterionic chelator compound. The number of polyethylene glycol units in the flexible linker may be varied to achieve the desired spacing between the zwitterionic chelator core and functional targeting regions of the compound while preserving the chelation properties of the zwitterionic framework.

In some cases, the flexible linker may comprise one to three polyethylene glycol units connecting the zwitterionic chelator to targeting vectors. A single polyethylene glycol unit may provide a shorter spacer length between the zwitterionic core and targeting moiety, while two or three polyethylene glycol units may provide progressively longer spacer lengths. The selection of one, two, or three polyethylene glycol units may allow for fine-tuning of the molecular geometry to optimize binding interactions with the target binding site while maintaining the zwitterionic properties and metal coordination stability of the zwitterionic chelator.

Alternatively, the flexible linker may comprise aminocarboxylic acid residues (for example, aminohexanoic acid) that serve as flexible spacer elements between the zwitterionic chelator and targeting vectors and also permit use of solid-phase synthesis. Aminocarboxylic acid residues may provide both flexibility and the ability to form hydrogen bonds with surrounding water molecules or protein residues. The aminocarboxylic acid residues may be selected based on their chain length and conformational properties to ensure compatibility with the zwitterionic chelator framework.

The flexible linker may comprise aminocarboxylic acid residues with varying carbon-chain lengths to adjust the distance between the zwitterionic chelator core and the target-binding targeting moiety. Shorter carbon-chain lengths may result in more compact molecular conformations that keep the zwitterionic chelator in close proximity to the targeting region, while longer carbon-chain lengths may allow for extended conformations. The carbon-chain length of the aminocarboxylic acid residues may be selected to position the zwitterionic chelator at the appropriate distance from the targeting moiety while preserving the chelator's coordination environment.

The length and composition of the flexible linker may be tuned to ensure that the zwitterionic chelator and any aromatic moieties are positioned within the target binding region at a distance typically on the order of 10-12 Å from the target anchor. This distance range may correspond to the spatial separation between the S1/S1′ cleft where the target anchor binds and the region where the zwitterionic chelator can be accommodated without interfering with target binding. The 10-12 Å separation may allow for simultaneous engagement of the targeting site and proper positioning of the target chelator without introducing unfavorable steric interactions that could compromise metal coordination.

The flexible alignment provided by the flexible linker may enhance binding affinity by allowing the zwitterionic chelator to adopt conformations that maximize favorable interactions with target binding sites while maintaining optimal chelator geometry. The flexibility may also maintain overall linker solubility and may prevent the formation of rigid conformations that could interfere with binding, cellular uptake processes, or the zwitterionic properties of the zwitterionic chelator system

The present disclosure leverages a modular approach that significantly enhances the efficiency and versatility of synthesizing final drug compounds. This modular strategy is particularly advantageous as it facilitates the use of standard solid-phase peptide synthesis (SPPS) techniques. By employing this approach, the invention allows for the precise and efficient assembly of peptide sequences, which are integral to the development of therapeutic agents. The compatibility with SPPS not only streamlines the synthesis process but also ensures high purity and yield of the final drug products. This innovation thus represents a significant advancement in the field of pharmaceutical manufacturing, enabling the scalable production of complex peptide-based therapeutics.

### Imaging Methods

In one aspect, the invention provides for methods of biomedical imaging of tissues or cells in a biological sample. In particular embodiment, the invention encompasses a method for detecting or imaging one or more cancer cells in a biological sample.

In some embodiments, the method is suitable for imaging of abnormal, but not malignant, tissue, such as defects of the musculoskeletal system using FAP as a targeting vector or vascular system using cRGD as a targeting vector.

In some embodiments, the method is suitable for imaging of cancer, tumor or neoplasm. In a further embodiment, the cancer is selected from eye or ocular cancer, rectal cancer, colon cancer, cervical cancer, prostate cancer, breast cancer and bladder cancer, oral cancer, benign and malignant tumors, stomach cancer, liver cancer, pancreatic cancer, lung cancer, corpus uteri, ovary cancer, prostate cancer, testicular cancer, renal cancer, brain/cns cancer (e.g., gliomas), throat cancer, skin melanoma, acute lymphocytic leukemia, acute myelogenous leukemia, Ewing's Sarcoma, Kaposi's Sarcoma, basal cell carinoma and squamous cell carcinoma, small cell lung cancer, choriocarcinoma, rhabdomyosarcoma, angiosarcoma, hemangioendothelioma, Wilms Tumor, neuroblastoma, mouth/pharynx cancer, esophageal cancer, larynx cancer, lymphoma, neurofibromatosis, tuberous sclerosis, hemangiomas, and lymphangiogenesis.

In some embodiments, the cancer cells are adult solid tumor cells or pediatric solid tumor cells. Non-limiting examples of such cells include melanoma cells, neuroblastoma cells, lung cancer cells, adrenal cancer cells, colon cancer cells, colorectal cancer cells, ovarian cancer cells, prostate cancer cells, liver cancer cells, subcutaneous cancer cells, squamous cell cancer cells, intestinal cancer cells, retinoblastoma cells, cervical cancer cells, glioma cells, breast cancer cells, pancreatic cancer cells, Ewings sarcoma cells, rhabdomyosarcoma cells, osteosarcoma cells, retinoblastoma cells, Wilms' tumor cells, and pediatric brain tumor cells.

In particular embodiments, the cancer cells are prostate cancer cells.

In some embodiments, the biological sample is part or all of a subject. In some embodiments, the biological sample is obtained from a subject.

In particular embodiments, the method includes the steps of (a) contacting the biological sample with one or more of the zwitterionic metal chelators described above, wherein the zwitterionic metal chelator is coordinated to a metal atom capable of being detected by one or more conventional scanning methods.

In some embodiments, the compound is administered by parenteral, intranasal, sublingual, rectal, or transdermal delivery. In some such embodiments, the compound is administered intravenously. In some embodiments, the compound is administered intratumorally.

In some embodiments, the tumor or cell is found in a subject. The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal (non-human) subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. In some embodiments, the subject is human. In other embodiments, the subject is non-human.

In particular embodiments, the subject is a human.

The methods of imaging tissue or cells include the following basic steps:
(a) contacting the tissue or cells with an imaging agent comprising a zwitterionic metal chelator; and
(b) imaging the tissue or cells using positron emission tomography (PET), single-photon emission computerized tomography (SPECT), or magnetic resonance imaging (MRI).

The imaging agent described herein is a substance to that can be used to image tissues or cells, such as those of a living organism, for purposes of diagnosis, therapy, image-guided surgery, and the like. In some embodiments, the organism is a mammal, such as a human.

The imaging agents described herein generally has improved “signal-to-background ratio” (SBR) compared to presently known imaging agents. The improvement in SBR is believed to be a result of improved in vivo properties due to “charge-balancing.” SBR is a measure of the intensity of the signal obtained from a target (peak signal) over the measure of the intensity of the signal obtained nearby the target (background signal), the target being the tissues or cells targeted by the imaging agent. SBR measurements can be readily obtained through routine measurement procedures. Higher SBR values are more desirable, resulting in greater resolution of the imaged tissues. In some embodiments, the imaging agents achieve an SBR of at least about 1.1 (i.e., peak signal is at least 10% over background). In further embodiments, the imaging agents achieve an SBR of at least about 1.2, at least about 1.3, at least about 1.4, at least about 1.5, at least about 1.6, at least about 1.7, at least about 1.8, at least about 1.9, or at least about 2.0. In yet further embodiments, the imaging agents achieve an SBR of about 1.1 to about 50, about 1.5 to about 30, about 2.0 to about 20, about 2.0 to about 5.0, or about 5.0 to about 10.

The zwitterionic metal chelators of the invention generally have good solubility in substantially neutral aqueous media, and in particular, blood and blood serum. In some embodiments, the imaging agent has a solubility in 10 mM HEPES solution, pH 7.4, of at least about 10 μM. In further embodiments, the imaging agent has a solubility in 10 mM HEPES solution, pH 7.4, of at least about 15 M at least about 20 μM, at least about 25 μM, at least about 30 μM, at least about 40 μM, or at least about 50 μM.

The zwitterionic metal chelators of the invention has significant improvements with regard to stability over time, allowing for dramatically improved operability and use for imaging and mapping. Similarly, the stability of the imaging agent allows for increased accuracy during surgery as the signal does not degrade over time. This is particularly important for complexes of Gd that are used as contrast for MRI. It has been established that Gd accumulates for long periods of time due to either instability in its metal chelator and/or inefficient elimination from the body. Zwitterionic metal chelators of Gd solve this problem by maximizing stability of the metal complex while maximizing rapid elimination from the body through renal clearance.

In another aspect, the disclosure encompasses a method of measuring and/or monitoring the effectiveness of various biological functions of a subject. In particular embodiments, the method provides for measuring the effectiveness of hepatic function, renal function, or blood pooling in a subject. In such embodiments, the zwitterionic metal chelators may be used with or without the addition of a targeting vector. The method includes the step of administering to a subject in need of measurement of a biological function with a quantifiable amount of one or more of the zwitterionic metal chelators described above; imaging the subject using positron emission tomography (PET), single-photon emission computerized tomography (SPECT), or magnetic resonance imaging (MRI); and determining the amount of the zwitterionic metal chelator present in the biological function being observed.

### Treatment, Diagnosis, and Monitoring Methods

### Cancerous Conditions

In another aspect, the disclosure encompasses a method for inhibiting the proliferation or growth of malignant or non-malignant cells. The method includes the step of contacting one or more cells with an effective amount of one or more of the zwitterionic metal chelators described above, wherein the metal atom is a radioactive metal isotope known to emit ionizing radiation in a form that would result in the death of cells that take up the analogs.

Non-limiting examples of metal isotopes that are used include Lu-177, Y-90, Ho-166, Re-186, Re-188, Cu-67, Au-199, Rh-105, Ra-223, Ac-225, As-211, Pb-212, and Th-227.

In some embodiments, the method is performed in vivo, ex vivo, or in vitro.

In some embodiments, the malignant cells are adult solid tumor cells or pediatric solid tumor cells. Non-limiting examples of such cells include melanoma cells, neuroblastoma cells, lung cancer cells, adrenal cancer cells, colon cancer cells, colorectal cancer cells, ovarian cancer cells, prostate cancer cells, liver cancer cells, subcutaneous cancer cells, squamous cell cancer cells, intestinal cancer cells, retinoblastoma cells, cervical cancer cells, glioma cells, breast cancer cells, pancreatic cancer cells, Ewings sarcoma cells, rhabdomyosarcoma cells, osteosarcoma cells, retinoblastoma cells, Wilms' tumor cells, and pediatric brain tumor cells.

In another aspect, the disclosure encompasses a method of diagnosing cancer in a subject. The method includes one or more of the imaging/detection steps outlined above. In the method, the biological sample is obtained from, part of, or all of a subject. If cancer cells are detected or imaged in the method steps, the subject is diagnosed with cancer. In certain embodiments, the method of diagnosing cancer in a subject is followed by a step of treating the subject diagnosed with cancer with a cancer therapy. In some embodiments, the cancer therapy is, surgery, chemotherapy or radiotherapy.

In some embodiments, the cancer that is diagnosed is an adult solid tumor or a pediatric solid tumor. Non-limiting examples of such cancer include melanoma, neuroblastoma, lung cancer, adrenal cancer, colon cancer, colorectal cancer, ovarian cancer, prostate cancer, liver cancer, subcutaneous cancer, squamous cell cancer, intestinal cancer, retinoblastoma, cervical cancer, glioma, breast cancer, pancreatic cancer, Ewings sarcoma, rhabdomyosarcoma, osteosarcoma, retinoblastoma, Wilms' tumor, and pediatric brain tumors.

In still another aspect, the disclosure encompasses a method of monitoring the efficacy of a cancer therapy in a human subject. The method includes performing one or more of the imaging/detection steps outlined above at two or more different times on the biological sample, wherein the biological sample is obtained from, part of, or all of a subject. The change in strength of the signals characteristic of the metal isotope between the two or more different times is correlated with the efficacy of the cancer therapy.

In another aspect, the disclosure encompasses a method of treatment of abnormal, but not malignant, tissue, such as defects of the musculoskeletal system using FAP as a targeting vector or vascular system using cRGD as a targeting vector. The method includes the step of contacting one or more abnormal cells with an effective amount of one or more of the zwitterionic metal chelators described above, wherein the metal atom is a radioactive metal isotope known to emit ionizing radiation in a form that would result in a therapeutic effect on the cells that take up the analogs.

In another aspect, the disclosure encompasses a method of treating a cancer by administering an effective amount of a therapeutic agent comprising a zwitterionic metal chelator complex and a pharmaceutically acceptable carrier or excipient. The method comprises a step to diagnose the cancer and a step of administering the therapeutic agent to a subject determined to be in need thereof,
wherein the step to diagnose the cancer comprises:
   contacting cells, tissues or organs of a subject with an imaging agent,
   imaging the cells, tissues, or organs of the subject using positron emission tomography (PET), single-photon emission computerized tomography (SPECT), or magnetic resonance imaging (MRI), and
   diagnosing the cancer in the cells tissues, or organs of the subject based on imaging data collected;
and wherein the metal atom complexed to the zwitterionic metal chelator is:
   a radioactive metal isotope known to emit ionizing radiation that results in the death of cells that take up the analogs;
   or a non-radioactive metal that is capable of releasing cytotoxic radiation upon irradiation with alpha emission, beta emission, neutron capture, or a combination thereof.

In still another aspect, the disclosure encompasses a method of treating a rare or childhood cancer by administering an effective amount of a therapeutic agent comprising a zwitterionic metal chelator complex and a pharmaceutically acceptable carrier or excipient. In such aspects, the therapeutic agent may further comprise one or more targeting vectors selected from the group consisting of cRGD, PSMA, FAP, octreotide, a bombesin analog, or a homomeric or heteromeric oligomers formed from their combination.

In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC(SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin. In addition, the metal atom complexed to the zwitterionic metal chelator is:
a radioactive metal isotope known to emit ionizing radiation that results in the death of cells that take up the analogs;
or a non-radioactive metal that is capable of releasing cytotoxic radiation upon irradiation with alpha emission, beta emission, neutron capture, or a combination thereof.

In particular embodiments, the rare or childhood cancer is: Acinic cell carcinoma, ACTH-secreting tumor, Actinic keratosis, Adamantinoma, Adenoid cystic carcinoma, Alveolar soft part sarcoma, Ampullary cancer, Angiosarcoma, Appendix (appendiceal) neuroendocrine (carcinoid) tumor, Askin tumor—a type of Ewing tumor (Ewing sarcoma), Bartholin gland cancer, Basaloid squamous cell carcinoma of the anus, Bowen disease, Bronchioloalveolar carcinoma, Carcinoid tumor, Carcinoma of the ampulla of Vater, Cardiac angiosarcoma, Castleman disease, Cholangiocarcinoma, Choriocarcinoma, Choroid plexus tumor, Chondrosarcoma, Chordoma, Chromophobe renal cell carcinoma, Clear cell sarcoma, Craniopharyngioma, Dermatofibrosarcoma protuberans, Desmoid tumor, Desmoplastic small round cell tumor, Dysgerminoma, Embryonal carcinoma, Endodermal sinus tumor, Endometrial stromal sarcoma, Ependymoma, Epithelial appendix (appendiceal) cancer, Epithelial-myoepithelial carcinoma, Epithelioid hemangioendothelioma (EHE), Epithelioid sarcoma, Essential thrombocythemia, Esthesioneuroblastoma (olfactory neuroblastoma), Extra-cranial malignant rhabdoid tumor (MRT), Extranodal NK/T-cell lymphoma—nasal type, Fallopian tube cancer, Fibrolamellar carcinoma, Fibromatosis, Fibromyxoid sarcoma (Evans' tumor), Fibrosarcoma Folliculotropic mycosis fungoides, Ganglioglioma, Ganglioneuroblastoma, Gastric Adenocarcinoma and Proximal Polyposis of the Stomach (GAPPS), Gastrinoma, Gastroesophageal junction (GEJ) cancer, Gestational trophoblastic disease (GTD) (hydatidiform mole; gestational trophoblastic neoplasia), Germ cell tumor, Giant cell tumor of bone, Glucagonoma, Granulomatous slack skin, Heart cancer (cardiac angiosarcoma), Hemangioendothelioma, Hemangiosarcoma, Hepatobiliary cancer, Hepatoblastoma, Hepatocellular carcinoma, Hepatoma, Hereditary diffuse gastric cancer (HDGC), Hurthle cell cancer (oxyphil cell carcinoma), Insulinoma, Islet cell tumor, Keratoacanthoma, Klatskin tumor, Large cell neuroendocrine carcinoma, Leiomyosarcoma, Leydig cell tumor, Lip cancer, Liposarcoma, Lymphomatoid papulosis, Lymphoplasmacytic lymphoma, Malignant mesenchymoma, Malignant mixed mullerian tumor, Malignant peripheral nerve sheath tumor (MPNST), Malignant rhabdoid tumor of the kidney, Medulloepithelioma, Meningioma, Mesoblastic nephroma, Metaplastic cancer of the breast, Monoclonal gammopathy of undetermined significance (MGUS), Mouth cancer, Mucinous cystic neoplasm, Mucoepidermoid carcinoma, Muscle cancer (myosarcoma), Myoepithelial carcinoma, Mycosis fungoides, Myelofibrosis, Myxofibrosarcoma, Nephroblastoma, Neuroendocrine carcinoma of the skin, NUT carcinoma, Oat cell cancer, Occult primary cancer, Ocular or intraocular melanoma, Olfactory neuroblastoma (esthesioneuroblastoma), Oligodendroglioma, Oncocytic carcinoma, Ovarian small cell cancer, Paget disease, Pagetoid reticulosis, Paraganglioma, Parathyroid cancer, Periosteal osteosarcoma, Peripheral primitive neuroectodermal tumor (PPNET), Pheochromocytoma, Phyllodes tumor, Pineoblastoma, Plasmacytoma, Polycythemia vera, Polymorphous low-grade adenocarcinoma, Primary cutaneous lymphoma, Primary peritoneal carcinoma, Prolactinoma (lactotroph adenoma), Renal cell carcinoma, Sarcomatoid carcinoma (carcinosarcoma), Schwannoma, Sclerosing epithelioid fibrosarcoma, Sebaceous carcinoma, Seminoma, Sertoli cell tumor, Sezary syndrome, Sinus cancer, Skin adnexal tumors, Solid pseudopapillary neoplasm, Solitary fibrous tumor, Solitary plasmacytoma, Somatostatinoma, Spermatocytic seminoma, Spindle cell neoplasm, spindle cell tumor, spindle cell carcinoma, spindle cell sarcoma, Subcutaneous panniculitis-like T-cell lymphoma, Synovial sarcoma, T-cell lymphoma, Teratoma, Throat cancer, Thymoma, Tongue cancer, Tonsil cancer, Trabecular cancer, Translocation renal cell carcinoma, Transitional cell carcinoma (urothelial carcinoma), Undifferentiated pleomorphic sarcoma, Urachal cancer, Urethral cancer, Urothelial carcinoma (transitional cell carcinoma), Uterine cancer, Verrucous carcinoma, VIPoma, Vocal cord/voice box cancer, Womb cancer, or Yolk sac tumor.

### Non-Cancerous Conditions

In another aspect, the invention provides a method of treating a non-cancerous condition in a subject in need thereof, the method comprising: administering to the subject an effective amount of a therapeutic agent comprising a zwitterionic metal chelator complex and a pharmaceutically acceptable carrier or excipient. In certain embodiments, the metal atom complexed to the zwitterionic metal chelator is:
a radioactive metal isotope known to emit ionizing radiation that results in a therapeutic effect on the subject.
or a non-radioactive metal that is capable of releasing therapeutic radiation upon irradiation with alpha emission, beta emission, neutron capture, or a combination thereof.

In certain embodiments, the non-cancerous condition is a musculoskeletal disorders or a tissue hypertrophy disorder. In still other embodiments, the non-cancerous condition is myocardial infarction, atherosclerosis, or fibro-inflammatory disease (including, but not limited to interstitial lung disease, rheumatoid arthritis, liver fibrosis, and keloids).

In still other embodiments, the subject is a human.

### Efficacy Measurements of Biological Systems.

In another aspect, the disclosure encompasses a method of measuring the efficacy of a biological system of a subject. In particular embodiments, the biological system to be measured or monitored is the renal system, the hepatic system, or the blood pool.

In particular embodiments, the method of measuring the efficacy of a biological system includes:
(a) administering a quantifiable amount of a diagnostic agent comprising a zwitterionic metal chelator complex and a pharmaceutically acceptable carrier or excipient to the subject;
(b) imaging the subject with positron emission tomography (PET), single-photon emission computerized tomography (SPECT), or magnetic resonance imaging (MRI); and
(c) determining the amount of therapeutic agent present in the biological system being observed in the subject.

In a particular aspect, the disclosure encompasses a method of measuring the efficacy of renal function a subject. In particular embodiments, the method comprises:
(a) administering a quantifiable amount of a diagnostic agent comprising a zwitterionic metal chelator complex and a pharmaceutically acceptable carrier or excipient to the subject;
(b) imaging the subject with positron emission tomography (PET), single-photon emission computerized tomography (SPECT), or magnetic resonance imaging (MRI); and
(c) determining the amount of therapeutic agent present in the biological system being observed in the subject.

In another aspect, the invention provides a method of quantifying the glomerual filtration rate of a subject. In particular embodiments, the method comprises:
(a) administering a quantifiable amount of a diagnostic agent comprising a zwitterionic metal chelator complex and a pharmaceutically acceptable carrier or excipient to the subject; and
(b) determining the amount of diagnostic agent present in the blood and urine of the subject as a function of time using either measurements of each bodily fluid or imaging the subject with positron emission tomography (PET), single-photon emission computerized tomography (SPECT), or magnetic resonance imaging (MRI).

### Radiosurgery

Radiosurgery is a known method of treating targets in the body. During radiosurgery, the target is bombarded with a series of X-ray beams fired from various different positions and orientations by using a radiation delivery system, to affect the tumor biology using the cumulative radiation dose at the target. The radiation can be delivered invasively in conjunction with traditional scalpel surgery, or through a percutaneous catheter. CyberKnife™ (Accuray Inc.) and Trilogy™ (Varian Medical Systems) are two such radiation delivery systems. Advances in stereotactic surgery have provided increased accuracy in registering the position of tissue targeted for treatment and a radiation source. For example, see U.S. Pat. Nos. 6,351,662 and 6,402,762. Stereotactic radiosurgery systems may be commercially available from ACCURAY, INC. of Sunnyvale, Calif., and BRAINLAB. The Accuray Cyberknife™ stereotactic radiosurgery system has reportedly been used to provide targeted, painless, and fast treatment of tumors.

In one aspect, the invention provides a radiosurgical method for treating a patient body, the method comprising:
receiving a desired lesion pattern and planned radiation distribution;
administering an effective amount of a diagnostic agent comprising a zwitterionic metal chelator complex and a pharmaceutically acceptable carrier or excipient to the subject to effectively image the desired lesion pattern; and
performing surgery on the desired lesion pattern to treat the patient body.

In certain embodiments, the surgery can be using scalpel surgery or a stereotactic radiosurgery system to administer a cumulative radiation dose.

In other embodiments, the zwitterionic metal chelator of the diagnostic agent further comprises one or more targeting vectors wherein the one or more targeting vectors are cRGD, KUE, dPSMA-617, a GPI-derivative, a FAP-targeting molecule, octreotide, a bombesin analog, or homomeric or heteromeric oligomers formed from their combination. In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC(SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin.

In still other embodiments, the desired lesion pattern is received from a user interface of a treatment planning module. Such planning modules can be pre-programmed with specifications for various disease states and cancerous conditions. Alternatively, specifications for desired lesion patterns can be identified and produced using artificial intelligence data.

### Toxic Metals

The zwitterionic metal chelators described herein can also be used as a treatment for removal of toxic or excess metals in a subject. In such methods, the zwitterionic metal chelators are administered without a metal coordinated thereto. The chelators act to bind excess or toxic metals in the subject which are then cleared along with the chelators through the kidneys and the renal system. The use of zwitterionic metal chelators for this method allows for faster removal of excess metals from a subject with increased clearance times and less damage to the liver and other organs.

As such, in one aspect, this disclosure encompasses a method for removing toxic or excess metals in a subject in need of such treatment, the method comprising:
(a) administering a therapeutically effective amount of a therapeutic agent to the subject,
wherein the therapeutic agent comprises a metal chelator having one or more zwitterionic groups which is not coordinated to a metal or metal isotope, and a pharmaceutically acceptable carrier or excipient.

In still other embodiments, the toxic or excess metals are lead, mercury, arsenic, cadmium, thallium, iron, zinc, chromium, manganese, aluminum, cobalt, selenium, beryllium, lithium, silver, or tin.

In still other embodiments, the toxic or excess metals are lead, mercury, arsenic, cadmium, thallium, iron, zinc, chromium, manganese, aluminum, cobalt, selenium, beryllium, lithium, silver, or tin.

### Other Applications—Site-Directed Labeling

The zwitterionic metal chelators of the invention can be conjugated to various biomolecules and targeting agents through site-directed labeling techniques. These conjugation methods allow for the precise attachment of the zwitterionic metal chelators to specific sites on target molecules while preserving the biological activity and binding properties of both the chelator and the target molecule.

The conjugation of zwitterionic metal chelators to monoclonal antibodies represents a particularly useful application of site-directed labeling. Monoclonal antibodies provide highly specific targeting capabilities for various disease states and cellular targets. In some embodiments, the zwitterionic metal chelators may be conjugated to monoclonal antibodies through lysine residues using NHS ester chemistry. The NHS ester group of the chelator reacts with primary amine groups on lysine residues to form stable amide bonds. In other embodiments, conjugation may occur through cysteine residues using maleimide chemistry, where the maleimide group reacts with sulfhydryl groups to form thioether linkages.

Site-specific conjugation techniques may be employed to achieve more controlled and homogeneous conjugates. In certain embodiments, engineered cysteine residues may be introduced at specific locations on the antibody to provide defined conjugation sites. Alternatively, unnatural amino acids containing reactive functional groups may be incorporated into the antibody during expression to enable site-specific attachment of the zwitterionic metal chelators.

The zwitterionic metal chelators may also be conjugated to other protein-based targeting agents including antibody fragments such as Fab, F(ab′)₂, scFv, diabodies, and nanobodies. These smaller protein constructs may offer advantages in terms of tissue penetration and clearance properties while maintaining specific binding capabilities.

The conjugation chemistry may also accommodate peptides, proteins, nucleic acids, carbohydrates, and synthetic polymers. For peptide conjugation, standard peptide coupling reagents such as carbodiimides (EDC, DCC), coupling agents (HATU, HBTU), and activating agents may be employed. Nucleic acid conjugation may utilize phosphoramidite chemistry or click chemistry approaches such as copper-catalyzed azide-alkyne cycloaddition (CuAAC) or strain-promoted azide-alkyne cycloaddition (SPAAC).

The site-directed labeling approach allows for the preparation of conjugates with improved pharmacokinetic properties, enhanced target specificity, and reduced off-target effects compared to non-specifically labeled constructs. The zwitterionic nature of the metal chelators may contribute to reduced non-specific binding and improved clearance properties of the resulting conjugates

### Other Applications—Agricultural and Chemical Processes

Zwitterionic metal chelators improve solubility and minimize non-specific interactions. It is believed that these improved properties result from the balancing of formal charges on the metal chelator, rendering a polyionic yet “charge-balanced” molecule having a net charge that is neutral or close to neutral, with an extended sphere of hydration and better isolation of the metal.

The zwitterionic metal chelators described herein can also be used in agricultural systems. In particular embodiments, zwitterionic metal chelators described herein can be used to supply micronutrients (trace metals such as iron, zinc, manganese, and copper) to various crops and plants. These micronutrients can help prevent drought and other diseases, particularly those caused by over-usage of certain fertilizers (particularly phosphorous fertilizer). Similarly, the zwitterionic metal chelators described herein can be used to supply various herbicidal formulations which are otherwise insoluble.

Similarly, the zwitterionic metal chelators described herein can also be used in chemical processes. As with the exposure to toxic metals described herein, certain chemical processes require the sequestration and removal of excess metals, such as metal catalysts. As such, the zwitterionic metal chelators described herein can also be used in such chemical process to remove insoluble metal impurities and/or unwanted metal components from the chemical process.

In particular, the zwitterionic metal chelators described herein have an advantage as compared to traditional metal chelators in the polarity of the zwitterionic molecules. As a result of this polarity, the zwitterionic metal chelators described herein can be used in aqueous solution, e.g. to increase solubility of a metal, and can then be precipitated from solution by addition of less polar solvents. This precipitation allows for the capture and subsequent disposal of undesired metals, —for example in capturing iron from wastewater (urban mining). In other embodiments, the zwitterionic metal chelators described herein which include sulfobetaines, precipitation can be induced by salt addition.

### Dosage Forms and Administration Methods

In the methods of the invention, the zwitterionic metal chelators are administered at a predetermined dosage. The predetermined dosage amount is not particularly limited provided that the predetermined dosage is administered at least a minimum amount capable of being cleared by the kidneys within twelve hours. In particular embodiments utilizing non-radioactive techniques, the predetermined dosage is 0.5 mg/kg of body weight; 0.25 mg/kg of body weight; 0.1 mg/kg of body weight; 0.05 mg/kg of body weight; 0.01 mg/kg of body weight; 0.005 mg/kg of body weight; or 0.001 mg/kg of body weight. In other embodiments, the predetermined dosage is 5.0 mg; 2.5 mg, 1.0 mg; 0.75 mg; 0.5 mg; 0.25 mg; or 0.1 mg. In particular embodiments utilizing radioactive techniques, the dose will be limited by dose-limiting toxicity. In general, effective doses will be in the range of 0.1 mCi to 20 mCi for imaging, and 0.1-10 mCi/kg for therapy.

In some embodiments, a detectably effective amount of the zwitterionic metal chelators of the presently disclosed methods is administered to a subject. In accordance with the presently disclosed subject matter, “a detectably effective amount” of the imaging agent is defined as an amount sufficient to yield an acceptable image using equipment which is available for clinical use. A detectably effective amount of the agent may be administered in more than one injection. The detectably effective amount of the imaging agent can vary according to factors such as the degree of susceptibility of the individual, the age, sex, and weight of the individual, idiosyncratic responses of the individual, the dosimetry, and instrument and film-related factors. Optimization of such factors is well within the level of skill in the art.

The administration of the agent of the invention can be by any means described herein and as generally acceptable to the patient and one of ordinary skill in the art. In particular, the administration of the charge-balanced imaging agent is intravenous.

The predetermined target amount is dependent on a variety of factors including, but not limited to the type of tumor or condition to be observed and the location of any cells desired to be imaged. As such, the predetermined amount is set by one of ordinary skill in the art prior to administration of the agent. Such factors include, but are not limited to, the determined dosage amount, the height, weight, age, body mass index, and gender of the patient or any combination thereof.

In particular embodiments utilizing non-radioactive techniques, when the predetermined dosage amount is between about 2.5 mg and about 5.0 mg or greater, the predetermined target amount is 50% of the pre-determined dosage amount. In other embodiments, when the predetermined dosage amount is between about 0.5 mg and about 2.5 mg, the predetermined target amount is 60% of the pre-determined dosage amount. In still other embodiments, when the predetermined dosage amount is about 0.5 mg or less, the predetermined target amount is 80% of the pre-determined dosage amount.

Any route of administration may be suitable for administering the disclosed agents to a subject. In one embodiment, the disclosed zwitterionic metal chelators may be administered to the subject via intravenous injection. In another embodiment, the disclosed zwitterionic metal chelators may be administered to the subject via any other suitable systemic deliveries, such as parenteral, intranasal, sublingual, rectal, or transdermal administrations.

In another embodiment, the disclosed zwitterionic metal chelators may be administered to the subject via intraperitoneal injection or IP injection.

In another embodiment, the disclosed zwitterionic metal chelators may be administered to the subject via intratumoral injection.

For treatment of non-malignant or malignant tissues using neutron capture therapy, the subject is bombarded with neutrons at the appropriate energy after administration of the (typically targeted) zwitterionic chelator and adequate time for biodistribution, binding, and clearance of unbound dose.

The disclosure also includes methods of using pharmaceutical compositions comprising one or more of the disclosed zwitterionic metal chelators in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, auto-injector devices or suppositories; for parenteral, intranasal, sublingual or rectal administration, or for administration by inhalation or insufflation.

For preparing solid compositions such as tablets, the principal active ingredient is mixed with a pharmaceutically acceptable carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture for a zwitterionic metal chelator of the present invention, or a pharmaceutically acceptable salt thereof. When referring to these preformulation compositions as homogeneous, it is meant that the active ingredient is dispersed evenly throughout the composition so that the composition may be easily subdivided into equally effective unit dosage forms such as tablets, pills and capsules. This solid pre-formulation composition is then subdivided into unit dosage forms of the type described above containing from 0.1 to about 500 mg of the active ingredient of the present invention. Typical unit dosage forms contain from 1 to 100 mg, for example, 1, 2, 5, 10, 25, 50 or 100 mg, of the active ingredient. The tablets or pills of the novel composition can be coated or otherwise compounded to provide a dosage affording the advantage of prolonged action. For example, the tablet or pill can comprise an inner dosage and an outer dosage component, the latter being in the form of an envelope over the former. The two components can be separated by an enteric layer which, serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the zwitterionic metal chelators may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

The disclosed zwitterionic metal chelators are particularly useful when formulated in the form of a pharmaceutical injectable dosage, including in combination with an injectable carrier system. As used herein, injectable and infusion dosage forms (i.e., parenteral dosage forms) include, but are not limited to, liposomal injectables or a lipid bilayer vesicle having phospholipids that encapsulate an active drug substance. Injection includes a sterile preparation intended for parenteral use.

Five distinct classes of injections exist as defined by the USP: emulsions, lipids, powders, solutions and suspensions. Emulsion injection includes an emulsion comprising a sterile, pyrogen-free preparation intended to be administered parenterally. Lipid complex and powder for solution injection are sterile preparations intended for reconstitution to form a solution for parenteral use. Powder for suspension injection is a sterile preparation intended for reconstitution to form a suspension for parenteral use. Powder lyophilized for liposomal suspension injection is a sterile freeze dried preparation intended for reconstitution for parenteral use that is formulated in a manner allowing incorporation of liposomes, such as a lipid bilayer vesicle having phospholipids used to encapsulate an active drug substance within a lipid bilayer or in an aqueous space, whereby the formulation may be formed upon reconstitution. Powder lyophilized for solution injection is a dosage form intended for the solution prepared by lyophilization (“freeze drying”), whereby the process involves removing water from products in a frozen state at extremely low pressures, and whereby subsequent addition of liquid creates a solution that conforms in all respects to the requirements for injections. Powder lyophilized for suspension injection is a liquid preparation intended for parenteral use that contains solids suspended in a suitable fluid medium, and it conforms in all respects to the requirements for Sterile Suspensions, whereby the medicinal agents intended for the suspension are prepared by lyophilization. Solution injection involves a liquid preparation containing one or more drug substances dissolved in a suitable solvent or mixture of mutually miscible solvents that is suitable for injection.

Solution concentrate injection involves a sterile preparation for parenteral use that, upon addition of suitable solvents, yields a solution conforming in all respects to the requirements for injections. Suspension injection involves a liquid preparation (suitable for injection) containing solid particles dispersed throughout a liquid phase, whereby the particles are insoluble, and whereby an oil phase is dispersed throughout an aqueous phase or vice-versa. Suspension liposomal injection is a liquid preparation (suitable for injection) having an oil phase dispersed throughout an aqueous phase in such a manner that liposomes (a lipid bilayer vesicle usually containing phospholipids used to encapsulate an active drug substance either within a lipid bilayer or in an aqueous space) are formed. Suspension sonicated injection is a liquid preparation (suitable for injection) containing solid particles dispersed throughout a liquid phase, whereby the particles are insoluble. In addition, the product may be sonicated as a gas is bubbled through the suspension resulting in the formation of microspheres by the solid particles.

The parenteral carrier system includes one or more pharmaceutically suitable excipients, such as solvents and co-solvents, solubilizing agents, wetting agents, suspending agents, thickening agents, emulsifying agents, chelating agents, buffers, pH adjusters, antioxidants, reducing agents, antimicrobial preservatives, bulking agents, protectants, tonicity adjusters, and special additives.

The present invention also provides packaged pharmaceutical compositions comprising a pharmaceutical acceptable carrier and a zwitterionic metal chelator of the invention. In certain embodiments the packaged pharmaceutical composition will comprise the reaction precursors necessary generate the zwitterionic metal chelator of the invention upon combination with a metal or radiolabeled precursor.

The invention also permits the zwitterionic metal chelator to be provided in a non-radioactive “kit” form comprised of the non-radioactive zwitterionic chelator/targeting vector molecule, a preparation apparatus (for example, a hot plate), and quality control devices (for example, a test strip or equivalent). The user would add the radioactive metal to create the final injected drug immediately before use as an imaging agent and/or therapeutic agent. In this way, the kit can be manufactured and shipped in a non-radioactive form, and the radioactive metal is added at the site. The non-radioactive zwitterionic chelator/targeting vector molecule may be provided in solution or in lyophilized form. When the imaging agent and carrier of the kit are in lyophilized form, the kit may optionally contain a sterile and physiologically acceptable reconstitution medium such as water, saline, buffered saline, and the like.

The kit may provide a zwitterionic metal chelator of the invention in solution or in lyophilized form, and these components of the kit of the invention may optionally contain stabilizers such as NaCl, silicate, phosphate buffers, ascorbic acid, gentisic acid, and the like. Additional stabilization of kit components may be provided in this embodiment, for example, by providing the reducing agent in an oxidation-resistant form.

Determination and optimization of such stabilizers and stabilization methods are well within the level of skill in the art. When the targeting molecule/chelating agent of this embodiment are in lyophilized form, the kit may optionally contain a sterile and physiologically acceptable reconstitution medium such as water, saline, buffered saline, and the like. The amounts of unlabeled targeting molecule/chelating agent, auxiliary molecule, and reducing agent in this embodiment are optimized in accordance with the methods for making the cardiovascular imaging agent set forth above. Radionuclides may be combined with the unlabeled targeting molecule/chelating agent and the reducing agent for a time and at a temperature sufficient to chelate the radionuclide to the targeting molecule/chelating agent, and the imaging agent thus formed is injected into the patient.

Imaging agents of the invention may be used in accordance with the methods of the invention by one of skill in the art. Images can be generated by virtue of differences in the spatial distribution of the imaging agents which accumulate at a site. The spatial distribution may be measured using any means suitable for the particular label, for example, a gamma camera, a PET apparatus, a SPECT apparatus, and the like. The extent of accumulation of the imaging agent may be quantified using known methods for quantifying radioactive emissions. A particularly useful imaging approach employs more than one imaging agent to perform simultaneous studies.

Generally, the diagnostic compositions are administered in doses effective to achieve the desired signal strength to enable detection. Such doses can vary, depending upon the organs or tissues to be imaged, and the imaging equipment being used. For example, Zeheer et al., Nature Biotechnology, 19, 1148-1154 (2001) uses 0.1 mol/kg as a dose for IRDye78 conjugates in vivo. The diagnostic compositions can be administered to a patient systemically or locally to the organ or tissue to be imaged, and then the patient is subjected to the imaging procedure.

Preferably, a detectably effective amount of the imaging agent of the invention is administered to a subject. In accordance with the invention, “a detectably effective amount” of the imaging agent of the invention is defined as an amount sufficient to yield an acceptable image using equipment which is available for clinical use. A detectably effective amount of the imaging agent of the invention may be administered in more than one injection. The detectably effective amount of the imaging agent of the invention can vary according to factors such as the degree of susceptibility of the individual, the age, sex, and weight of the individual, idiosyncratic responses of the individual, the dosimetry. Detectably effective amounts of the imaging agent of the invention can also vary according to instrument and film-related factors. Optimization of such factors is well within the level of skill in the art.

The amount of imaging agent used for diagnostic purposes and the duration of the imaging study will depend upon the radionuclide used to label the agent, the body mass of the patient, the nature and severity of the condition being treated, the nature of therapeutic treatments which the patient has undergone, and on the idiosyncratic responses of the patient. Ultimately, the attending physician will decide the amount of imaging agent to administer to each individual patient and the duration of the imaging study.

### EXAMPLES

### Example 1: Imaging of Organisms

For in vivo characterization, 40 pmol/g (average 10 nmol) of a zwitterionic metal chelator complex can be injected IV into 25 g athymic nude mice harboring xenograft human tumors. Simultaneous color video and positron emission tomography (PET), single-photon emission computerized tomography (SPECT), or magnetic resonance tomography (MRT) can be acquired pre-injection, every 1 sec for the first 20 sec then every 1 min for 2 h. Camera acquisition can be held constant (typically 100 msec) and chosen to ensure that all intensity measurements are within a linear range. Blood can be sampled at 0, 1, 2, 5, 10, 15, 30, 60, and 120 min via tail vein. Intensity-time curves for all major organs and tissues can be quantified. The peak intensity and time can be determined for each tumor/tissue/organ, along with the intensity in each at 1 h post-injection.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

Those skilled in the art will recognize, or be able to ascertain using no more than routine experimentation, many equivalents to the specific embodiments of the invention described herein.

Such equivalents are intended to be encompassed by the following claims.

### REFERENCES

REFERENCES

| Year | DOI | Title | Authors | Citation |
|---|---|---|---|---|
| 2014 | 10.1002/ | Synthesis of 1,4,7,10-tetra- | Kriemen et al. | Chem. Asian J. 2014, 9 (8), |
| | asia.201402250 | azacyclododecan-1,4,7,10-tetra- | | 2197-2204. |
| | | azidoethylacetic acid (DOTAZA) and | | |
| | | related “clickable” DOTA | | |
| | | derivatives. | | |
| 2017 | | Poster - P342 | Henry et al. | J. Label Compd Radiopharm, |
| | | Targeted Zwitterionic Radioligands | | 2017: 60 (Suppl. 1): S111-S640 |
| | | for Improved Molecular Imaging | | |
| 2019 | 10.1021/ | Nonradioactive Cell Assay for the | Holzapfel et al. | J. Med. Chem., 2019, 62, |
| | acs.jmedchem.9b01606 | Evaluation of Modular Prostate- | | 10912-10918 |
| | | Specific Membrane Antigen | | |
| | | Targeting Ligands via Inductively | | |
| | | Coupled Plasma Mass Spectrometry | | |
| 2022 | 10.1002/ | Solution Structure and Relaxivity of | Holzapfel et al. | Eur. J. Inorg. Chem. 2022, |
| | ejic.202200432 | Ln-DOTXAZA Derivatives. | | e202200432 |
| 2015 | 10.1002/ | Synthesis and Structural Analysis of | Kriemen et al. | Eur. J. Inorg. Chem. 2015, |
| | ejic.201500789 | 1,4,7,10-tetra-azacyclododecan- | | 5368-5378 |
| | | 1,4,7,10-tetra-azidoethylacetic | | |
| | | acid (DOTAZA) Complexes | | |
| 2023 | 10.1002/ | Synthesis of Modular | Outzen et al. | ChemMedChem 2023, |
| | cmdc.2003000112 | Desferriozamine Analogues and | | e202300112 |
| | | Evaluation of Derivatives for | | |
| | | Zirconium Complexation | | |
| 2019 | DOI: 10.1097/ | Physicochemical and | Robic et al. | J. Invest Radiol 2019; 54: |
| | RLI.0000000000000563 | Pharmacokinetic Profiles of | | 475-484 |
| | | Gadopiclenol: A New Macrocyclic | | |
| | | Gadolinium Chelate With High T1 | | |
| | | Relaxivity | | |
| 2019 | DOI: 10.1002/ | Efficient Synthesis of a Family of | Leygue et al. | Eur. J. Org. Chem. 2019; |
| | ejoc.201900280 | Bifunctional Chelators Based on | | 2899-2913 |
| | | the PCTA[12] Macrocycle | | |
| | | Suitable for Bioconjugation [symbol] | | |
| 2026 | Submitted | Zwitterionic MRI contrast agents | Spickschen | Journal of Materials Chemistry |
| | | with enhanced relaxivity, | et al. | B |
| | | stability, and reduced renal | | |
| | | retention | | |
| 2025 | DOI: 10.2967/ | Versatile Bifunctional PYTA | Cheveau et al. | J Nucl Med. 2025 |
| | jnumed.125.270234 | Derivatives for 225Ac | | September; 66(9): 1396-1399. |
| | | Radiolabeling: A Comparison to | | |
| | | Gold Standards | | |

[symbol]

indicates data missing or illegible when filed

The contents of all patent, patent applications, and publications cited herein are incorporated herein by reference in their entireties.

1. A zwitterionic metal chelator complex comprising a metal chelator having one or more zwitterionic groups and a metal or metal isotope selected from the group consisting of a radionuclide, a label, a paramagnetic metal and a heavy metal.

2. The zwitterionic metal chelator complex according to claim 1, wherein the metal chelator is a derivative of DOTP.

3. The zwitterionic metal chelator complex according to claim 1, further comprising one or more targeting vectors.

4. The zwitterionic metal chelator complex according to claim 3, wherein the one or more targeting vectors are cRGD, KUE, a GPI-derivative, PSMA-617, FAPI, octreotide, a GPI-derivative, a bombesin analog, or a homo- or hetero-dimer formed from their combination.

5. The zwitterionic metal chelator complex according to claim 1, wherein the metal chelator is a derivative of DOTP and the metal or metal isotope is Pd, Pb, Zr, Cu, Ga, In, Y, Gd, Lu, Ac or Tb.

6. The zwitterionic metal chelator complex according to claim 2, wherein the metal chelator is a derivative of DOTP and wherein the zwitterionic metal chelator of the zwitterionic metal chelator complex has the formula: 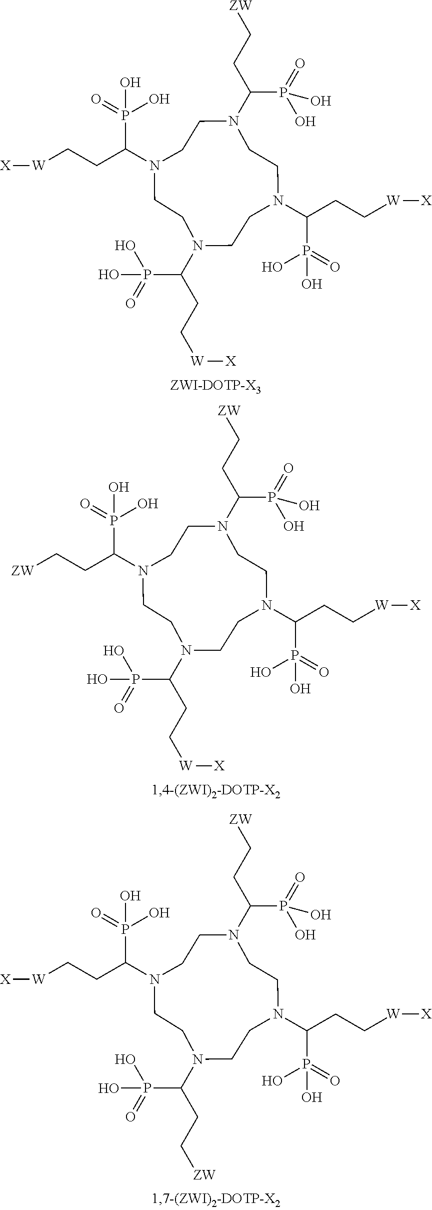 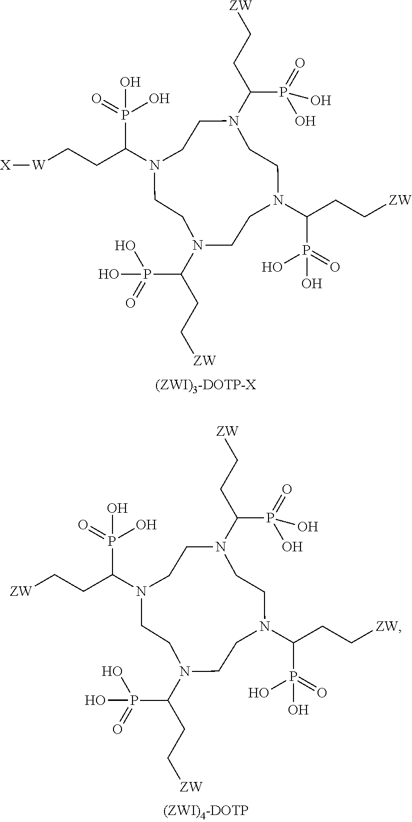
wherein ZW represents a zwitterionic group; each instance of W and Y independently represent a linking group, and each instance of X represents a reactive group or targeting vector.

7. An imaging agent comprising a zwitterionic metal chelator complex according to claim 1.

8. A method of imaging cells, tissues, or organs, the method comprising:
(a) contacting cells with an imaging agent according to claim 7; and
(b) imaging the cells, tissues, or organs using positron emission tomography (PET), single-photon emission computerized tomography (SPECT), or magnetic resonance imaging (MRI).

9-12. (canceled)

13. A therapeutic agent comprising a zwitterionic metal chelator complex according to claim 1 and a pharmaceutically acceptable carrier or excipient.

14. A method of treating a cancerous condition in a subject in need thereof, the method comprising:
contacting cancer cells in the subject with an effective amount of a therapeutic agent according to claim 13,
wherein the metal atom complexed to the zwitterionic metal chelator is:
a radioactive metal isotope known to emit ionizing radiation that results in the death of cells that take up the analogs;
or a non-radioactive metal that is capable of releasing cytotoxic radiation upon irradiation with alpha emission, beta emission, neutron capture, or a combination thereof.

15. The method of treating a cancerous condition in a subject in need thereof according to claim 14, wherein the metal atom complexed to the zwitterionic metal chelator is a non-radioactive metal that is capable of releasing cytotoxic radiation upon irradiation with alpha emission, beta emission, neutron capture, or a combination thereof: the method further comprising a step of irradiating the tumor cells using alpha emission, beta emission, neutron capture, or a combination thereof.

16-19. (canceled)

20. A method of treating a non-cancerous condition in a subject in need thereof, the method comprising:
administering to the subject an effective amount of a therapeutic agent according to claim 13,
wherein the metal atom complexed to the zwitterionic metal chelator is:
a radioactive metal isotope known to emit ionizing radiation that results in a therapeutic effect on the subject.
or a non-radioactive metal that is capable of releasing therapeutic radiation upon irradiation with alpha emission, beta emission, neutron capture, or a combination thereof.

21. The method of treating a non-cancerous condition in a subject in need thereof according to claim 20, wherein the non-cancerous condition is a musculoskeletal disorders or a tissue hypertrophy disorder.

22. (canceled)

23. A diagnostic agent comprising a zwitterionic metal chelator complex according to claim 1 and a pharmaceutically acceptable carrier or excipient.

24. A method of measuring the efficacy of a biological system of a subject, the method comprising:
(a) administering a quantifiable amount of a diagnostic agent according to claim 23 to the subject;
(b) imaging the subject with positron emission tomography (PET), single-photon emission computerized tomography (SPECT), or magnetic resonance imaging (MRI); and
(c) determining the amount of therapeutic agent present in the biological system being observed in the subject.

25. The method of a measuring the efficacy of a biological system in a subject according to claim 24, wherein the biological system is the renal system, the hepatic system, or the blood pool.

26. (canceled)

27. A method of quantifying the glomerual filtration rate of a subject, the method comprising:
(a) administering a quantifiable amount of a diagnostic agent according to claim 23 to the subject;
(b) determining the amount of diagnostic agent present in the blood and urine of the subject as a function of time using either measurements of each bodily fluid or imaging the subject with positron emission tomography (PET), single-photon emission computerized tomography (SPECT), or magnetic resonance imaging (MRI).

28. A method for removing toxic or excess metals in a subject in need of such treatment, the method comprising:
(a) administering a therapeutically effective amount of a therapeutic agent according to claim 13 to the subject,
wherein the therapeutic agent comprises a metal chelator having one or more zwitterionic groups which is not coordinated to a metal or metal isotope, and a pharmaceutically acceptable carrier or excipient.

29. A radiosurgical method for treating a patient body, the method comprising:
receiving a desired lesion pattern and planned radiation distribution;
administering an effective amount of a diagnostic agent according to claim 23 to the subject to effectively image the desired lesion pattern;
performing surgery on the desired lesion pattern to treat the patient body.

30. The radiosurgical method according to claim 29, wherein the zwitterionic metal chelator of the diagnostic agent further comprises one or more targeting vectors
wherein the one or more targeting vectors are cRGD, PSMA-617, FAPI, octreotide, a bombesin analog, or a homo- or hetero-dimer formed from their combination.

31-35. (canceled)


SEQUENCE LISTING (6 sequences)

SEQ ID NO: 1 - protein, 9 aa, Homo sapiens
CGQKRTRGC

SEQ ID NO: 2 - protein, 12 aa, Homo sapiens
HTMYYHHYQHHL

SEQ ID NO: 3 - protein, 9 aa, Homo sapiens
CRKRLDRNC

SEQ ID NO: 4 - protein, 6 aa, Homo sapiens
CSPGAK

SEQ ID NO: 5 - protein, 8 aa, Homo sapiens
FCFWKTCT

SEQ ID NO: 6 - protein, 14 aa, Homo sapiens
XQRLGNQWAVGHLM